(19) United States
(12) Patent Application Publication
(10) Pub. No.: US 2002/0168365 A1
(43) Pub. Date: Nov. 14, 2002

(54) Method for modulating granzyme B uptake and for identifying modulators thereof

(76) Inventors: Chris Bleackley, Edmonton (CA); Bruce Motyka, Edmonton (CA)

Correspondence Address: MORRISON & FOERSTER LLP, 755 PAGE MILL RD, PALO ALTO, CA, 94304-1018, US

(21) Appl. No.: 10/000,789

(22) Filed: Oct. 25, 2001

Related U.S. Application Data
(60) Provisional application No. 60/243,509, filed on Oct. 25, 2000.

(51) Int. Cl. A61K 39/395; A61K 48/00; A61K 38/17

(52) U.S. Cl. 424/146.1; 514/44; 514/12

(57) ABSTRACT

The present invention relates to the identification of CI-MPR as the receptor responsible for binding and internalization of grB into cells. In addition, the present invention relates to the binding and/or internalization of grB by CI-MPR as an event necessary to enable grB-mediated apoptosis in cells. The present invention further identifies the binding and/or internalization of grB through CI-MPR as a target which is used by cancer cells to evade the grB-mediated apoptosis and clearing by the immune system. The present invention further provides assays to identify modulators of grB binding to CI-MPR and/or internalization of grB thereby. In addition, the invention provides methods to correct a disease or condition associated with a defect in grB binding to CI-MPR and/or internalization of grB thereby, which comprises an administration of an effective amount of a modulator of grB-CI-MPR interaction.

### CROSS-REFERENCE TO RELATED APPLICATIONS

This application claims the priority benefit of the provisional patent application U.S. Ser. No. 60/243,509, filed Oct. 25, 2000, which is incorporated by reference in its entirety.

### FIELD OF THE INVENTION

The present invention relates to the binding of granzyme B (grB) to the membrane of cells, to the internalization of grB thereinto, and its effect on cellular processes. More specifically, the present invention relates to a method for modulating granzyme B uptake and for identifying modulators thereof. In particular, the present invention relates to a modulation of apoptosis and proliferation by a modulation of grB uptake by cells.

### BACKGROUND OF THE INVENTION

Two mechanisms have been proposed for the induction of apoptosis in virus-infected and tumor cells by CTLs (reviewed by Shresta et al., 1998). Cytotoxic T lymphocytes (CTLs) are effector cells of the human immune system that have lytic capability and are critical in the recognition and elimination of virus-infected and tumor cells (Atkinson et al., 1995). CTLs kill target cells by inducing them to die by apoptosis, an induced and ordered process in which the cell actively participates in bringing about its own demise. Cells undergoing apoptosis exhibit distinctive morphological and biochemical changes (Berke, 1995). These changes include a pronounced decease in cell volume, disruption of mitochondrial transmembrane potential and the release of cytochrome c, phosphatidylserine externalization from the inner to outer leaflet of the plasma membrane and DNA fragmentation. Following these events, an apoptotic cell sheds tiny membrane-bound apoptotic bodies, which are quickly phagocytosed by macrophages.

The first involves interaction of the membrane-bound Fas ligand on CTLs with the Fas receptor on the surface of target cells. Engagement of the Fas receptor results in activation of caspase 8 which is then capable of activating downstream effector caspases, either by direct proteolytic cleavage or indirectly through activation of Bid and release of mitochondrial cytochrome c (Honglin et al., 1998). These caspases are in turn responsible for the inactivation of a variety of cellular proteins leading to eventual cell death.

The latter involves the introduction of granzymes, members of the serine proteinase family, into target cells by granule-mediated exocytosis of cytolytic molecules. When antigen-specific CTLs associate with appropriate target cells, the two cell types interact and undergo conjugate formation.

The granule-exocytosis model has evolved since its original enunciation by Henkart (Henkart, 1985). Initially one of the granule proteins, perforin, was thought to be the major effector of CTL-induced death. However, it then became apparent that target cells were dying through an apoptotic mechanism that involved damage of DNA (Duke et al., 1989). The factor responsible was purified and identified (Shi et al., 1992a, 1992b) as a known cytotoxic cell protease (CCP1) (Lobe et al., 1986), now referred to as granzyme B (grB) (Masson and Tschopp, 1987). In a complementary approach, it was shown that cells were endowed with the ability to induce membrane damage and DNA fragmentation when they were transfected to express grB and perforin (Nakajima et al., 1995). Finally, a role for grB in the DNA damage pathway was firmly established with experiments using CTL from grB-deficient mice (Heusel et al., 1994; Shresta et al., 1995).

In the revised model of killing, it was envisaged that perforin polymerized in response to calcium and inserted into the target cell membrane to create a channel through which granzyme could pass. Once the proteinase is inside the target cell cytoplasm the apoptotic program is initiated through the activation of caspases, the cysteine proteases responsible for execution of the cell death pathway (reviewed in Wolf and Green, 1999). Initially it was shown in vitro that caspase 3 was a substrate for grB (Darmon et al., 1995; Martin et al., 1996) and now similar results have been observed for a large number of the caspases (Darmon and Bleackley, 1998). Of note, caspases 8 and 3 have been shown to be direct substrates for grB in intact cells (Atkinson et al., 1998).

The perforin channel model has been widely accepted, although there is very little experimental evidence to prove polyperforin can transport a macromolecule of ˜30 kDa such as grB (Browne et al., 1999). Replication-deficient adenovirus (AD) can substitute for perforin and was reported to facilitate uptake of grB and induce apoptosis (Froelich et al., 1996). Subsequent studies indicated that grB uptake occurs independently of AD (Shi et al., 1997; Pinkoski et al., 1998). Cells with internalized grB showed no morphological signs or biochemical markers of apoptosis until they were subsequently treated with perforin or AD. We hypothesized that grB was taken up by receptor-mediated endocytosis and required perforin or AD to mediate its release into the cytoplasm and hence gain access to critical substrates such as the caspases.

A fundamental prediction from such a hypothesis is the existence of a cell surface receptor that can bind and internalize grB. There thus remains a need to identify this receptor in order to validate the receptor-mediated endocytosis of grB hypothesis and to disprove the widely accepted model by which grB is predominantly internalized by a perforin-dependent mechanism.

In addition, there remains a need to demonstrate the importance of grB uptake in apoptosis mediated by granule-purified grB or by CTL.

Once identified, the level of expression of the receptor responsible for grB internalization on cells might be correlatable with a resistance or sensitivity thereof to the immune system. There thus also remains a need to assess whether the level of expression (and/or functionality) of the receptor responsible for grB internalization in cells plays a role in their resistance/sensitivity to host defenses.

There also remains a need to provide the means of correcting a disease or condition in a cell or animal associated with a disturbance of the binding pathway, comprising grB internalization and biological activity within the cell.

There also remains a need to provide a diagnosis or prognosis of a disease based on a defect in grB internalization.

The present invention seeks to meet these and other needs.

The present description refers to a number of documents, the content of which is herein incorporated by reference in their entirety.

### SUMMARY OF THE INVENTION

In an alternate scheme to the widely accepted perforin-dependent internalization of grB, the present invention discloses the existence of a cell surface receptor, the cation-independent mannose-6-phosphate/insulin-like growth factor 2 receptor (CI-MPR) that binds and internalizes grB in a perforin-independent manner. Of note, as it will be demonstrated below, expression of this receptor was necessary for the efficient apoptosis of target cells by granule-purified grB and CTL in vitro.

We hypothesized that some cancer cells, such as breast cancer cells, may have acquired the ability to evade the effects of grB as a result of a perturbation in the grB-CI-MPR interaction and/or endocytosis. This would play a critical role in the escape of tumor cells from host defenses. We examined MPR expression, grB binding and grB-mediated apoptosis in four breast cancer cell lines (T47-D, MDA-MB-231, SK-BR-3 and MCF-7) in order to determine if there was a correlation between MPR expression and grB-mediated killing. As will be demonstrated below, the instant invention demonstrates that all cancer cell lines tested have evolved the means to evade granule purified grB-mediated apoptosis.

The present invention therefore concerns the identification of the receptor responsible for the binding and internalization of grB in cells. The present invention demonstrates that the major pathway for grB internalization in cells is through its binding and internalization with CI-MPR. An internalization of grB in a perforin-dependent fashion, while not the major pathway, might still occur in certain cell lines or under particular conditions, however.

The present invention further relates to the demonstration that there is a direct correlation between binding and uptake of grB by cells and the triggering of apoptosis thereinto. Thus, the present invention enables methods to modulate grB-mediated apoptosis in cells, comprising a modulation of the binding of grB to the receptor and/or an internalization of grB in cells. In one particular embodiment, the present invention exemplifies significant prevention of the binding and/or uptake of grB to its MPR receptor, comprising a diphosphorylation of grB, a co-incubation of the MPR receptor with a ligand thereof (e.g. M6P or M6P-containing molecule), or co-incubation with soluble CI-MPR). In a particular embodiment of the present invention, such inhibition of grB binding and/or uptake by a cell significantly inhibits the grB-mediated apoptosis pathway thereinto.

The invention in addition relates to a method to induce apoptosis in a target cell comprising increasing the CI-MPR mediated uptake of grB in said target cell. In one embodiment, the method involves increase in the expression of CI-MPR and/or increase in the externalization of CI-MPR to the cell surface in the target cells. In another embodiment, the method of inducing apoptosis in a target cell involves the incubation of an apoptosis-inducing amount of grB with a target cell which expresses the CI-MPR receptor or analog or derivative thereof which enables binding and uptake of the grB in the target cell, thereby triggering the induction of apoptosis thereinto.

In addition, the invention relates to a method of modulating the clearance of transplanted allogeneic cells in an animal comprising the modulation of the grB binding and uptake in the transplanted allogeneic cells, wherein a significant binding and uptake of grB by the transplanted allogeneic cell is associated with a rejection thereof, while a reduction or absence of binding and internalization of grB is associated with an increased survival of the transplanted cells.

The Applicant was the first to identify the MPR and more particularly the CI-MPR receptor as the receptor responsible for binding and internalization of grB in cells. The Applicant was also the first to demonstrate the importance of receptor expression for binding and of capture of grB and for its associated effect on apoptosis as well as the critical importance of MPR expression for cell death. In addition, the Applicant was the first to demonstrate that in the absence of grB receptor, the allogeneic cells survived while grB receptor expressing allogeneic cells were rejected in in vivo experiments.

In view of the reported importance of grA for graft-versus-host disease after bone marrow transplantation, and the survival of CI-MPR cells, the present invention suggests for the first time that CI-MPR is the receptor responsible for uptake of both grA and grB.

Before the present invention, a receptor for internalization of grB had not been identified. In addition and consequently, prior to the present invention, the level and/or functionality of this receptor on cell physiology and more particularly on the triggering of the apoptotic pathway or on evading the immune system had yet to be disclosed.

In view of the intricate regulation that operates at CI-MPR (e.g. it acts as a receptor for grB, grA, IGF-II, TGF∃ CD26, retinoic acid and some viruses), and the fact that it mediates lyposomal targeting of a heterogeneous population of over 40 soluble acid hydrolases which differ in N-linked oligosaccharides, phosphorylation state, position of M6P moiety and size (Marron-Terada et al. 1988, J. Biol. Chem. 273:22358-22366), the present invention provides the mean to dissect the structure function relationship of CI-MPR and to elucidate how the regulation of binding and internalization of these different substrates impact the internalization of grB and hence the cell physiology.

The present invention also provides a screening method for identifying compounds capable of enhancing or inhibiting at least one of a) an interaction between grB and the MPR receptor; b) grB-mediated apoptosis; and c) CTL-dependent killing of cells, which involves contacting cells which express MPR (e.g. CD-MPR and/or CI-MPR, preferably the latter) with a candidate compound, assaying a biological activity, and comparing the biological activity to a standard biological activity, the standard being assayed when contact is made in the absence of the candidate compound; whereby an increased biological activity over the standard indicates that the compound is an agonist and a decreased biological activity over the standard indicates that the compound is an antagonist.

Additional aspects of the invention relate to methods for treating an animal and more particularly a human in need of either an increased or decreased level of grB internalization in the body comprising administering to such an animal a composition comprising a therapeutically effective amount of either a modulator of grB internalization and/or grB and/or MPR polypeptides of the invention. One non-limiting model disease in which an animal would be in need of an increased level of internalization of grB is cancer. One non-limiting model disease or condition in which an animal would be in need of a decreased level of internalization of grB is a disease in which an evasion of the immune system is desired. Examples thereof include auto-immune diseases and upon transplantation in the animal and more particularly a human.

The present invention also relates to a method to diagnose or prognose in an animal a disease or condition associated with an increased or decreased level of internalization of grB in cells associated with this disease or condition, comprising determining the level of at least one of the CI-MPR and CD-MPR receptor at the surface of the cells. In a particular embodiment of the invention, this determining is carried-out with a ligand specific to at least one of these receptors and preferably CI-MPR. In another embodiment, the expression level of CD-MPR and/or CI-MPR is determined. For such a determination, nucleic acid probes can be used. In a particular embodiment, the level of membrane bound CI-MPR is compared to its internalized level.

The findings listed in Example 1 suggest that the CI-MPR plays an important role in CTL killing. A cell expressing low or no CI-MPR is thus expected to be more capable of evading the immune system. This is corroborated by the correlation between grB internalization and cancer described in Example 2. It would thus be useful to determine the levels of CI-MPR (and CD-MPR since it can interfere with grB internalization) on the surface of, for instance, tumor cells. As the experiments shown in Example 2 suggest, the absence of CI-MPR surface expression, the over-surface expression of CD-MPR, or an inability of the CI-MPR molecules to be externalized to the cell surface correlate to a poor clinical prognosis. Such a determination might enable the choice of a better suited therapeutic regimen. In one embodiment in which a cancer cell for example is shown to have a low level of expression of CI-MPR or of externalization of CI-MPR to the cell surface, the treatment regimen can be adapted accordingly. For example, should grB internalization be increased in such a cancer cell, a vaccine targeting a tumor receptor on this cancer cell could be used to trigger a CTL-dependent killing thereof.

In accordance with the present invention, there is also provided a method for identifying, from a library of compounds, a compound with therapeutic effect on a disease or condition associated with a perturbed internalization of grB, comprising providing a screening assay which comprises a measurable biological activity of CI-MPR or involving a CI-MPR-grB interaction or interacting domains thereof; contacting the screening assay with a test compound; and detecting if the test compound modulates the biological activity of CI-MPR or of the interaction between CI-MPR and grB; wherein a test compound which modulates the biological activity is a compound with this therapeutic effect. Non-limiting examples of such diseases or conditions associated with a perturbation of internalization of grB include cancer and auto-immune disorders.

Also provided within the present invention is a compound having therapeutic effect on a disease or condition associated with a perturbed internalization of grB identified by a method comprising: providing a screening assay which comprises a measurable biological activity of CI-MPR or involving a CI-MPR-grB interaction or interacting domains thereof; contacting the screening assay with a test compound; and detecting if the test compound modulates the biological activity effected by CI-MPR or by the interaction between CI-MPR and grB, wherein a test compound which modulates the biological activity is a compound with this therapeutic effect. Non-limiting examples of such diseases or conditions associated with a perturbation of internalization of grB include cancer and auto-immune disorders.

Yet in another embodiment, the present invention relates to an assay to screen for drugs for the treatment and/or prevention and/or prognosis of a disease or condition associated with a perturbation of grB internalization such as, for example, cancer or conversaly auto-immune diseases. In a particular embodiment, such assays can be designed using cells from patients having such a disease or condition. These cells harboring recombinant vectors can enable an assessment of the functionality of a recombinant CI-MPR (whether wild-type or mutated) to identify agonists or antagonists of the CI-MPR function in internalizing grB, whether in terms of expression, activity or cellular localization. Non-limiting examples of assays that could be used in accordance with the present invention include cis-trans assays similar to those described in U.S. Pat. No. 4,981,784.

In accordance with one embodiment of the present invention, there is therefore provided, a method for identifying an agent that modulates CI-MPR-dependent internalization of granzyme B in a cell comprising contacting a CI-MPR, or fragment thereof in the presence or absence of a candidate compound and assaying a biological function of the CI-MPR, or fragment thereof, wherein a modulator of the CI-MPR-dependent internalization of granzyme B is selected when the biological function is measurably different in the presence of the candidate agent as compared to in the absence thereof.

In accordance with another embodiment of the present invention, there is also provided a method for identifying an agent that modulates CI-MPR-dependent internalization of granzyme B in a cell comprising contacting a CI-MPR, or fragment thereof with a mannose 6-phosphate-containing molecule in the presence or absence of a candidate compound and assaying a biological function of said CI-MPR, wherein a modulator of the CI-MPR-dependent internalization of granzyme B is selected when the biological function is measurably different in the presence of the candidate agent as compared to in the absence thereof.

In accordance with yet another embodiment of the present invention, there is provided a method of increasing granzyme B internalization in a cell comprising increasing the level and/or activity of CI-MPR on the outer membrane of the cell.

In accordance with an additional embodiment of the present invention, there is provided a method of inhibiting a disease or disorder in an individual associated with an enhanced internalization of granzyme B (grB), comprising administering to the individual an effective amount of an agent which inhibits grB binding to the cation-independent mannose 6-phospate receptor (CI-MPR) and/or internalization of grB thereby.

As briefly noted above, cells undergoing apoptosis display characteristic features such as DNA fragmentation and externalization of phosphatidylserine onto surface of plasma membrane that can be monitored using a variety of assays. Extensive DNA degradation is a characteristic event which often occurs in the early stages of apoptosis. DNA strand breaks can be detected by enzymatic labeling of the free 3 OH termini with X-dUTP by terminal deoxynucleotidyl transferase (TUNEL end labeling) (Guide to Cell Proliferation and Apoptosis Methods, 2000, Roche Diagnostics Corporation). The labeled DNA is subsequently analyzed by flow cytometry. One advantage of flow cytometry is that it is automatable and thus amenable to high throughput screening.

A number of other markers can be monitored to assess early, intermediate or late events in cellular apoptosis. Such markers and assays are well-known in the art and hence the present invention should not be limited to the assays exemplified in the present invention. A number of these assays provide the advantage of enabling automation and/or high-throughput screening.

While the present invention demonstrates and exemplifies the effect of grB binding and internalization on apoptosis and immune evasion and methods of modulating same, the present invention should not be so limited. Indeed, the present invention enables the correction of any disease or condition in which the physiological role of grB is compromised by a defect in binding and/or internalization thereof through CI-MPR. Thus, the present invention relates to diseases or conditions in which grB-CI-MPR interaction is inhibited or stimulated or in which a defect in CI-MPR has been reported. Non-limiting examples of such diseases or conditions include of course cancer as well as auto-immune diseases, viral infections, tissue or organ transplantation and multiple sclerosis.

While the present invention has demonstrated that breast cancer cells have devised a number of strategies to inhibit or lower grB internalization (or the activity of grB), the present invention should not be so limited. Indeed, other cancer cells are believed to use at least one of these strategies to override the grB-mediated apoptotic pathway. Non-limiting examples of such cancer cells include pancreas cells, melanoma cells, lymphomas, ovarian cells and neural cells. In fact, abundant evidence link CI-MPR mutations to tumors. While some of these mutations still result in CI-MPR being expressed, the ability thereof to bind M6P-ligands or IGF-II was found to be compromised in many instances. The teachings of the present invention together with such studies therefore provide the framework to test the level and/or functionality of CI-MPR (membrane bound or internalized) in such cancer cells and use such cells or CI-MPR therefrom in assays or methods of the present invention. What follows are selected, non-limiting examples in which CI-MPR mutations have been documented.

Microsatellite instability (MSI) has been shown to be present in 15% of sporadic colorectal carcinomas. Several genes (including TGF beta RII, and IGFIIR) were shown to harbor repeats in their coding regions and are often somatically inactivated because of deletions causing frameshifts. IGF-II and its receptor have been suggested to play an important role in the development of hepatocarcinogenesis (hepatocellular carcinoma (HCC), since a mutation in the IGF-IIR/CI-MPR has been discovered in HCC. M6P/IGF2R has also been found to be mutated in squamous cell carcinoma of the lung. A lung adenocarcinoma cell line was found to have a point mutation in the CI-MPR. In mice we have identified murine squamous (SCC-VII) carcinoma cells, whose secretion of matrix-degrading cathepsins is attributable to a deficiency in the mannose 6-phosphate/insulin-like growth factor II receptor. The association between CI-MPR and disease is not limited to cancer since, for example, insulin-like growth factor II receptors which are present in human brain have been shown to be absent in astrogliotic plaques in multiple sclerosis.

In view of the teachings of the present invention, it is expected that a perturbation (quantitative or qualitative [i.e. mutation]) which affects positively or negatively binding or uptake of a given M6P-containing protein, will similarly affect binding or uptake of grB. It follows that it is within the scope of the present invention to assess grB binding or uptake through its interaction with CI-MPR with other M6P-containing proteins which interact with CI-MPR. Non-limiting examples of M6P-containing proteins include IGF-II, TGF∃, retinoic acid. Interactions with soluble acid hydrolases could also be monitored. Such enzymes as well as biological activities of CI-MPR are discussed in Marron-Terada et al. 1988, J. Biol. Chem. 273:22358-22366.

In addition, while the present invention has been exemplified with human grB and human CI-MPR, the present invention should not be so limited. Indeed, in view of the functional complementation showed by the human CI-MPR+ L-cells (mouse cells) originally deficient in CI-MPR expression and the in vivo mouse experiment shown in Example 1, and of the significant conservation of these sequences as well as those of downstream effectors of grB function, other mammalian sequences could be used in the methods and assays of the present invention. Indeed, it is shown herein that human CI-MPR when expressed in mouse cells can bind and internalize grB (ie. the CI-MPR+ cells mentioned in Example 1 (the human CI-MPR expressing mouse L cells deficient in endogenous CI-MPR)). Evidence is presented that mouse grB binds to and is internalized via the human CI-MPR (ie. in vitro CTL assays with murine CTLs and our CI-MPR+ targets). Similarly we observe that human grB binds to and is internalized by the murine CI-MPR. In fact, CI-MPR is found in the vast majority of higher eukaryotic cell types and from the comparison of the cDNA sequences from bovine, mouse, rat and human, it is clear that CI-MPR is highly conserved (Dahns, 1996, Biochem Soc. Trans. 24:136). The scope of the present invention thus also includes non-mammalian higher eukaryotic cell types.

Of note, there are two types of mannose 6-phosphate (M6P) receptors: the cation independent MPR (CI-MPR) also known as the insulin-like growth factor receptor II (IGF-IIR) and the cation-dependent MPR (CD-MPR). Both can bind grB, although the affinity toward CD-MPR is significantly lower than toward CI-MPR (see below).

The CD-MPR protein sequence are highly conserved from bovine to human (with about 92% identity overall). The CI-MPR protein homolog between rat and human is 83% overall with the functionally important regions thereof being highly conserved (see below). Because of the ease to purify CI-MPR from bovine, the bovine CI-MPR has been well studied (and sequenced). In many cases, the bovine CI-MPR cDNA has been introduced in an expresion vector in murine cells which lack endogenous CI-MPR expression and a functional implementation between bovine and murine demonstrated (Marron-Terada et al. 1988, J. Biol. Chem. 273:22358-22366).

### BRIEF DESCRIPTION OF THE DRAWINGS

Having thus generally described the invention, reference will now be made to the accompanying drawings, showing by way of illustration a preferred embodiment thereof, and in which:

FIG. 1 shows that the phosphatase treatment of grB inhibits its ability to bind at the cell surface and to mediate apoptosis. (A) Flow cytometric analysis of the binding of grB-biotin to Jurkat cells after prior treatment of the proteinase at 37° C. for 60 min with or without alkaline phosphatase (AP) in the presence or absence of phosphate buffer (PO₄) as described in Example 1. The percentage of positive staining cells (region markers not shown in the three-dimensional histogram) and the relative mean fluorescence intensity (MFI), shown in brackets, are indicated. Relative cell number is represented on the y axis. (B) DNA fragmentation and phosphatidylserine externalization as assessed by TUNEL and annexin V labeling, respectively, after a 3 hr treatment at 37° C. of Jurkat cells with 10 pfu/cell replication-deficient AD and 100 ng/ml grB that was or was not pretreated with alkaline phosphatase as outlined above. The percent specific positive labeling cells was determined by flow cytometric analysis as outlined in Example 1. The percentage of TUNEL/annexin V positive cells incubated without treatment or with only grB was under 8%. Data shown in (A) is representative of at least three experiments. Data shown in (B) is the mean±SD of three experiments.

FIG. 2 shows that grB binds to both the CI- and the CD-MPR but it is the expression of the CI-MPR that is required for grB-mediated apoptosis. (A) The binding of grB was compared with the level of expression of cell surface MPR using antisera specific for the CI- and CD-forms of the MPR. Wild-type L-cells and the L(Rec⁻) cell series (MPR-, CI-MPR⁺-, and CD-MPR⁺-cells) were treated as described above with grB-OG or with anti-MPR. All labeling steps were carried out at 4° C. Data is presented as the relative MFI as described in Example 1. (B) TUNEL and annexin V labeling of L cells and the L(Rec⁻) cell series after a 3 hr incubation with 100 ng/ml grB and AD. The percent specific positive labeling cells was determined by flow cytometric analysis. Data shown in (A) is representative of at least three experiments. Data shown in (B) is the mean±SD of at least three experiments.

FIG. 3 shows that M6P inhibits grB binding, uptake and apoptosis induction. (A) GrB-OG binding to CI-MPR⁺ cells in the presence or absence of M6P or D-mannose at different concentrations, or with 50 mM D-glucose or D-glucose-6 phosphate. Cells were labeled and incubated as previously described. Maximal binding was taken as the MFI of grB-OG binding to CI-MPR⁺ cells in the absence of monosaccharide. (B) Confocal analysis of CI-MPR⁺ cells incubated with grB-OG in the presence or absence of M6P. Cells were incubated for 20 min at 37° C. without grB-OG (a), or with grB-OG either in the absence (b), or presence of 20 mM M6P (c). (C) Jurkat cells were incubated for 3 hr with different concentrations of grB and AD following a 15 min incubation with different concentrations of M6P or related monosaccharides. Cells were then labeled using a TUNEL or annexin V assay and analyzed by flow cytometry. The percentage of TUNEL/annexin V positive cells after incubation with grB and AD with either 25 mM D-glucose 6-phosphate, D-mannose, or D-glucose was 60/41, 52/32, and 66/40, respectively. Data shown in (A-C) is representative of at least three experiments.

FIG. 4 shows the correlation of CTL-mediated target cell DNA fragmentation with CI-MPR expressions. A human CTL line was incubated with either CI-MPR⁺ or MPR⁻ target cells at E:T ratios of 4:1, 2:1, and 1:1. The percent specific ³H-thymidine-release (measure of DNA fragmentation) and ⁵¹Cr-release (measure of outer-membrane damage) of the target cells was determined after a 3- and 5-hr incubation, respectively. Data is the mean±SD of three experiments.

FIG. 5 shows the correlation of CI-MPR expression with allograft survival. CI-MPR⁺ and MPR⁻ cells (H-2^{k}-expressing) were used as donor cells for transplantation under the kidney capsule of allogeneic recipient BALB/c or SCID mice. Mice were sacrificed after 7- or 14-days and immunohistochemical labeling of the kidney capsule was performed using mAb specific for H-2^{k}, CD4, or CD8 as described in Example 1. Bars correspond to 10 μm. Data shown is representative of three experiments.

FIG. 6 shows that grB binding correlates to surface MPR expression. The binding of grB was compared with the level of expression of cell surface MPR using antisera specific for CI-MPR and CD-MPR and biotin labeled grB. Data is presented as the relative MFI as described in Example 2. Data shown is representative of three experiments.

FIG. 7 shows a reduction of CI-MPR expression in certain cell lines. Levels of expression of total CI-MPR was examined using antiserum specific for CI-MPR after cells were permeabilized allowing entry of antibody. Data is presented as the relative MFI as described in Example 2.

FIG. 8 shows the inhibition of grB binding by M6P. Flow cytometric analysis of the binding of grB-OG to cells after prior incubation with M6P at 37EC for 15 min. Data is presented as the relative MFI as described in Example 2. Data shown is representative of three experiments.

FIG. 9 shows the induction of apoptosis by grB and AD. Annexin V labeling of cells after a 3 hr incubation with grB (0.72, 3.6 and 18 nM) and AD (10 pfu per cell). The percent specific positive cells was determined by flow cytometric analysis. Data shown is the mean±SD of three experiments.

FIG. 10 shows the induction of apoptosis by grB and AD. TUNEL labeling of cells after a 3 hr incubation with grB (0.72, 3.6 and 18 nM) and AD (10 pfu per cell). The percent specific positive cells was determined by flow cytometric analysis. Data shown is the mean±of three experiments.

FIG. 11 shows that caspase 3 is processed in Jurkat and T47D cells following addition of grB and AD. Cells were incubated with grB (18 nM) and AD (10 pfu per cell) for 3 hr and caspase 3 cleavage was assessed by Western blotting analysis.

FIG. 12 shows that caspase 3 is processed in all cell lines except MCF7 following addition of staurosporine. Cells were incubated with staurosporine (2.5:M) and caspase 3 cleavage was assessed by Western blotting analysis.

FIG. 13 shows an alignment between human (GI4504611) and bovine (GI89650) cation-independent mannose-6-phosphate receptor, displaying 80% identity, 88%homology, wherein the GI number indicates an NCBI accession number.

FIG. 14 shows an alignment between human (GI14750582) and mouse (GI55857) granzyme B precursor displaying a 68% identity and 69% homology overall, wherein the GI number indicates an NCBI accession number.

FIG. 15 shows an alignment between human (GI12745007) and bovine (GI89649) cation-dependent mannose-6-phosphate receptor, displaying 92% identity and 94% homology, wherein the GI number indicates an NCBI accession number.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

Mammalian cells possess two mannose-6-phosphate receptors, the cation-dependent (CD-MPR) and the cation-independent receptor (CI-MPR), also known as the insulin-like growth factor receptor (IGF-IIR) (Sandholzer et al., 2000). The two MPRs have apparent molecular weights of 46 kDa and ˜270 kDa, respectively. Both MPRs are type I transmembrane glycoproteins and the extracytoplasmic domain of CD-MPR is homologous to each of the 15 repeating units in the extracytoplasmic domain of CI-MPR. CI-MPR can bind M6P-containing ligands at the cell surface and endocytose extracellular M6P-containing ligands. Ligands bind to the repeating units 3 and 9 and arginine residues appear to be involved in the binding (Marron-Terada et al. 1998 supra).

The serine proteinase granzyme B is crucial for the rapid induction of target cell apoptosis by cytotoxic T cells. Evidence that granzyme-B enter cells in a perforin-independent manner is now presented since its cell surface receptor, the cation-independent mannose 6-phosphate/insulin-like growth factor receptor (CI-MPR), has been identified. Inhibition of the granzyme B-CI-MPR interaction prevented granzyme B cell surface binding, uptake, and the induction of apoptosis. Significantly, expression of the CI-MPR was essential for cytotoxic T cell-mediated apoptosis of target cells in vitro and for the rejection of allogeneic cells in vivo. These results provide a novel target for immunotherapy and a potential mechanism used by tumors for immune evasion. To corroborate this immune evasion mechanism in tumors, cancer cells were chosen and tested. A correlation between the level or the activity of CI-MPR and cancer and/or apoptosis was identified. Strikingly, the present invention indeed demonstrates that carcinoma cell lines use several strategies to evade granule purified grB-mediated apoptosis.

The discovery of the grB-CI-MPR interaction is important for a number of reasons, one of which being that the receptor has previously been implicated as a tumor suppressor gene (Hankins et al., 1996). The receptor is involved in inactivation of IGF-2, a potent autocrine and paracrine mitogen which otherwise triggers mitogenic signals on IGF-1 receptors. It also activates TGF-β, an immunosuppressive agent, and regulates targeting of lysosomal enzymes, particularly procathepsins. Loss of heterozygosity at the M6P locus on chromosome 6q and somatic mutations in coding regions have been reported in breast and liver cancer and tumors of the gastrointestinal tract, endometrium and brain (Ouyang et al., 1997; Chappell et al., 1997; Sue et al., 1995; and De Souza et al., 1995).

Sequences

A number of homologs and orthologs of the nucleic acid and amino acid sequences of the present invention are known. The human CI-MPR cDNA and encoded protein are available in the NCBI database under accession number NM_{—}000867; SEQ ID NOs 1 and 2, respectively. The human cDNA for granzyme B and its encoded protein are known (see NCBI database, XM_{—}032600; SEQ ID NOS:3 and 4, respectively). Granzyme B (grB) has two N-linked glycosylation sites which can be modified to express the M6P-modification and hence enable internalization in cells. The human nucleic acid and amino acid sequences of CD-MPR are also available in the NCBI database under accession number NM_{—}002355; SEQ ID NOs 5 and 6, respectively. Sequences of the present invention are also available from other animal species. These can be found in different databases as commonly known. Non-limiting examples of such animals thereof include bovine, mouse, rat, chicken, frog and zebra fish. For example from NCBI the sequences of CI-MPR from mus musculus (AAA19568); bovine (A30788); vattus norvegicus (AAB03185); and gallus gallus (AAC59718) can be obtained. From NCBI, sequences of grB can be obtained from rat (A43520); mus musculus (CAA27715); and bos taurus (AAG28537). The sequences of CD-MPR have been obtained from many species including zebra fish (BF157978), frog (BI350257), and chicken (BI394729).

In order to provide a clear and consistent understanding of terms used in the present description, a number of definitions are provided hereinbelow.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning-A Laboratory Manual, Cold Spring Harbor Laboratories) and Ausubel et al. (1994, Current Protocols in Molecular Biology, Wiley, N.Y.).

The apoptotic pathway methods of monitoring same are well-known in the art. The “Guide to Cell Proliferation and Apoptosis Methods” (2000, Roche Diagnostics Corporation) is but one example of a reference that provide teachings to be used in the context of the present invention.

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA or RNA) for practicing the present invention may be obtained according to well known methods. Non-limiting examples thereof include CI-MPR, CD-MPR, grB, grA and caspase 3. While CI-MPR, grB or CD-MPR are preferred sequences (nucleic acid and proteins) in accordance with the present invention, and especially the human sequences thereof, the invention should not be so limited. Indeed, in view of the significant conservation of the genes throughout evolution of the sequences which can be used to practice the invention, sequences from different animal species, and preferably mammalian species, could be used in the assays of the present invention. One non-limiting example is the mouse CI-MPR ortholog protein which shows 93% homology with its human counterpart. The significant conservation of the sequences of the present invention will be shown further below.

As used herein, the term “physiologically relevant” is meant to describe interactions which have relevance to their natural setting and more particularly in vivo. Encoding portions of the proteins encoded by these nucleic acids, which interact in order to mediate grB binding, grB internalization and/or grB-mediated apoptosis, are also within the scope of the present invention.

The term “ligand” is used herein to refer broadly to an agent which binds to one of the proteins of the present invention. In a particularly preferred embodiment, the ligands bind to CI-MPR or to grB. Non-limiting examples of ligands of CI-MPR, for example include antibodies thereto, kinases, grB, IGF-II, M6P, soluble acid hydrolases and the like. A number of ligands for CI-MPR has thus been identified. In addition, a number of specific domains thereof have been determined as being the interacting domains with particular ligands. For example, IGF-II binds to domain 11 of the CI-MPR (Marron-Terada et al. 1998 supra), whereas mannose 6-phosphorylated proteins bind to domain 3 and/or domain 9 of the CI-MPR. Nevertheless it is known that IGF-II blocks the binding and uptake of M6P-expressing proteins—perhaps through a steric hindrance mechanism. Competition experiments using IGF-II have shown that same does not inhibit M6P uptake by CI-MPR. However, an upregulation of M6P uptake in the presence of IGF-II has been reported. In fact, the stimulation of expression of the CI-MPR gene has been shown following IGF-II stimulation. IGF-II, parts or analogs thereof could thus be tested in the assays of the present invention. In its latent form, the TGF-beta cytokine binds to CI-MPR in a manner independent of the M6P-binding domains (domain 3 and 9) of CI-MPR. TGF-beta can also inhibit binding/uptake of M6P-proteins. LIF and the HSV glycoprotein D bind via the M6P-binding sites of the CI-MPR. CD26 and retinoic acid are examples of other ligands that bind the CI-MPR. CD26 is a protein that gets M6P phosphotylated upon T cell activation. In activated T cells, CD26 then crosslinks with itself when bound to its ligand CD26 and is then internalized via the CI-MPR. Retinoic acid can directly bind CI-MPR with high affinity, with a distinct binding site which is different from that used by IGF-II or M6P.

As used herein, “biological activity” or “CI-MPR biological activity” refers to any detectable biological activity of a gene or protein of the present invention. In particular, it refers in particular to a biological activity of CI-MPR, and grB as well as of CD-MPR, or grA gene or protein. More particularly, it refers to any detectable biological activity which is relevant to the interaction between CI-MPR and M6P-containing proteins of the present invention (e.g. grB). In view of the effect of CD-MPR on grB binding/internalization by CI-MPR, the term “biological activity” also relates to a detectable biological activity which is relevant to the interaction between CD-MPR and M6P-containing proteins. This includes any physiological function attributable to a CI-MPR or grB gene or protein. It can include for example the specific biological activity of CI-MPR enabling grB-mediated apoptosis. This includes measurement of apoptotic features in cells, but not limited to: 1) DNA fragmentation, 2) externalization of phosphotidyl serine onto the surface of the plasma membrane; and 3) activation of caspase 3. At a larger scale, CI-MPR biological activity includes in vitro or in vivo killing assays and transplantation rejection/maintenance, dependent on an internalization of grB, wherein changes in these activities caused by modulators of CI-MPR dependent grB internalization can be identified. The biological activity also includes lyposomal enzyme sorting. Non-limiting examples of biological activities are described in Marron-Terada et al. 1998 supra and include sorting of cathepsinD, secretion of αhexosaminodase and αglucuronidase, pentamannosyl phosphate-agarose affinity chromatography, and secretion of phosphorylated ligands. Non-limiting examples of measurements of these biological activities may be made directly or indirectly. In one embodiment, an indirect measure of CI-MPR biological activity is measured indirectly by measuring biological activity of grB. CI-MPR biological activity is not limited, however, to these most important biological activities herein identified. Biological activities may also include simple binding or pKa analysis of CI-MPR with compounds, substrates, interacting proteins, and the like. For example, by measuring the effect of a test compound on its ability to increase or inhibit such CI-MPR binding or interaction is measuring a biological activity of CI-MPR according to this invention. CI-MPR biological activity includes any standard biochemical measurement of CI-MPR such as conformational changes, phosphorylation status or any other feature of the protein that can be measured with techniques known in the art. CI-MPR biological activity also includes activities related to CI-MPR gene transcription or translation, or any biological activities of such transcripts or translation products. In addition, CI-MPR biological activity also include activities related to the externalization of CI-MPR on the surface of the cell membrane, or to the ratio of externalized to internalized CI-MPR in cells. Since the instant invention is concerned particularly with CI-MPR interaction with grB, biological activity of CI-MPR also includes assays which monitor binding and other biochemical measurements of grB. For clarity, in view of the number of ligands which interact or are internalized by CI-MPR (e.g. grA, TGF-α . . . ), biological activity also includes measurements using these ligands. Furthermore, the terminology “biological activity” also includes measurements based on the interaction of domains of interacting proteins of the present invention (e.g. at least one, preferably two of the M6P sites of grB with at least one of domains 3 and 9 of CI-MPR).

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised of the deoxyribonucleotides adenine (A), guanine (G), thymine (T) and/or cytosine (C), often in a double-stranded form, which can comprise or include a “regulatory element”, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. “Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5×Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al.,1989, supra).

Probes or sequences of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA), and preferably of DNA. Protein probes (e.g. labeled protein) can also be used.

The types of detection methods in which probes can be used include Southern blots (DNA detection), dot or slot blots (DNA, RNA), Northern blots (RNA detection) and in the case of labeled proteins, Westerns, West-Northerns or West-Southerns. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods. While they are usually in a single-stranded form, they can be in a double-stranded form and even contain a “regulatory region”.

As used herein, a “primer” defines an oligonucleotide which is capable of annealing to a target sequence, thereby creating a double stranded region which can serve as an initiation point for DNA synthesis under suitable conditions. Primers can be, for example, designed to be specific for certain alleles so as to be used in an allele-specific amplification system.

In a particular embodiment in which the level of expression of one of the interacting factors of the present invention need to be monitored (CI-MPR, CD-MPR or grB), a pair of primers is designed to specifically amplify a segment of one of the interacting factors of the present invention. This pair of primers is preferably derived from the nucleic acid sequence of CI-MPR, CD-MPR or grB or from sequences flanking these genes, to amplify a segment of CI-MPR, CD-MPR or grB. The design of primer pairs using the sequences of the CI-MPR, CD-MPR or grB nucleic acids molecules described hereinbelow are well-known in the art.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications, screening assays or the like.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation.

As used herein, the designation “functional derivative” denotes, in the context of a functional derivative of a sequence whether a nucleic acid or amino acid sequence, a molecule that retains a biological activity (either function or structural) that is substantially similar to that of the original sequence. This functional derivative or equivalent may be a natural derivative or may be prepared synthetically. In one embodiment, the derivative may be a homolog of one of the human sequences (or parts thereof) of the present invention. The derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. One non-limiting example of maintenance of biological activity includes a CI-MPR derivative which enables at least one of binding and internalization of grB. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. Conservative amino acid substitutions are well-known in the art. For example, amino acids considered to be within the same group are usually considered conservative substitutions. One such categorization includes six different groups of amino acids: aromatic, hydrophobic, polar, basic, acidic and small. Thus, for example, substituting a hydrophobic amino acid such as leucine by another, such as isoleucine, is considered a conservative substitution. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. All these methods are well known in the art.

As used herein, “chemical derivatives” is meant to cover additional chemical moieties not normally part of the subject matter of the invention. Such moieties could affect the physico-chemical characteristic of the derivative (e.g. solubility, absorption, half life, decrease of toxicity and the like). Such moieties are exemplified in Remington's Pharmaceutical Sciences (1980). Methods of coupling these chemical-physical moieties to a polypeptide or nucleic acid sequence are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. In one particular embodiment, a rational design of a molecule based on M6P and preferably a polyM6P (or polyM6P-containing protein or peptide) would be carried-out. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a perturbation, for example of the binding, internalization and/or downstream activity of grB. In one particular embodiment, the physiology of the cell is compromised by a defect in the triggering of grB-dependent apoptosis. Alternatively, the molecules identified in accordance with the teachings of the present invention find utility in the development of more efficient modulators of grB internalization in cells.

As used herein, agonists and antagonists of grB-MDR interaction, for example, also include potentiators of known compounds with such agonist or antagonist properties on IGF II receptor. In one embodiment, agonists can be detected by contacting the indicator cell with a compound or mixture or library of molecules for a fixed period of time and determining any one of biological features associated with the interaction assayed. In one particular embodiment, the effect of the compound or compounds in apoptosis is monitored.

The level of gene expression of the reporter gene (e.g. the level of luciferase, or β-gal, produced) within the treated cells can be compared to that of the reporter gene in the absence of the molecules(s). The difference between the levels of gene expression indicates whether the molecule(s) of interest modulates the level of expression of the reporter gene product expressed. The level of modulation (treated vs. untreated cells) provides a relative indication of the strength of that molecule(s) as a modulator.

An indicator cell in accordance with the present invention can be used to identify antagonists. For example, the test molecule or molecules are incubated with the host cell in conjunction with one or more agonists held at a fixed concentration. An indication and relative strength of the antagonistic properties of the molecule(s) can be provided by comparing the level of gene expression or activity of the encoded protein in the indicator cell in the presence of the agonist, in the absence of test molecules versus in the presence thereof. Of course, the antagonistic effect of a molecule can also be determined in the absence of agonist, simply by comparing the level of expression or activity of the reporter gene product in the presence and absence of the test molecule(s).

It shall be understood that the “in vivo” experimental model can also be used to carry out an “in vitro” assay. For example, cellular extracts from the indicator cells can be prepared and used in one of the aforementioned “in vitro” tests.

As used herein the recitation “indicator cells” refers to cells that express one of the nucleic acids of the present invention. In one particular embodiment, the indicator cell expresses CI-MPR (or domain which interacts with grB) and grB (or the domain thereof which interacts with CI-MPR), and wherein an interaction between these proteins or interacting domains thereof, is coupled to an identifiable or selectable phenotype or characteristic such that it provides an assessment of the interaction between same. Such indicator cells can be used in the screening assays of the present invention. In certain embodiments, the indicator cells have been engineered so as to express a chosen derivative, fragment, homolog, or mutant of these interacting proteins or domains. The cells can be yeast cells or higher eukaryotic cells such as mammalian cells (WO 96/41169). In view of the post-translational modifications of the factors involved in grB-mediated apoptosis, mammalian cells will be preferred for certain assays. Of course, yeast cells could also be used. In one such embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of the grB and CI-MPR interacting domains. Such an indicator cell could be used to rapidly screen at high-throughput a vast array of test molecules. In a particular embodiment, the reporter gene is luciferase or β-Gal. At least one of these two interaction domains of the present invention may be provided as a fusion protein. The design of constructs therefor and the expression and production of fusion proteins are well known in the art (Sambrook et al., 1989, supra; and Ausubel et al., 1994, supra). In one embodiment, both interaction domains are part of fusion proteins. In one such embodiment, the fusions are a LexA-grB fusion (DNA-binding domain-grB; bait) and a B42-CI-MPR fusion (transactivator domain-CI-MPR; prey). Such LexA-grB and B42-CI-MPR fusion proteins can be expressed in a yeast cell also harboring a reporter gene operably linked to a LexA operator and/or LexA responsive element.

Non limiting examples of fusion proteins include hemaglutinin fusions and Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art.

Bacterial OmpA and yeast Suc2 are two non limiting examples of proteins containing signal sequences. Of course, the signal sequences of the proteins of the present invention could also be used. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to one of its partners can be readily determined by using the teachings and assays of the present invention and the general teachings of the art.

As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner (in the case of grB, the interaction partner includes caspase 3 and CD-MPR, preferably CI-MPR) may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of grB-CI-MPR interaction.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, such an advantage might be rendered moot if both polypeptide tested directly interact. It will be understood that extracts from mammalian cells for example could be used in certain embodiments, to compensate for the lack of certain factors.

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art. Non-limiting examples of anti-sense which can be used in the context of the present invention include CD-MPR or CI-MPR antisense molecules.

This invention now establishes, for the first time, that CI-MPR, is directly responsible for binding and internalizing grB in animals and more particularly in humans. Further, this discovery strongly suggests that compounds which modulate the activity or level of CI-MPR, will have applicability for treating diseases or conditions associated with a deregulated internalization of grB, and notably cancer. It is therefore an object of this invention to provide screening assays using CI-MPR or parts thereof which can identify compounds which have therapeutic benefit for such diseases or conditions. This invention also claims those compounds, the use of these compounds in such diseases or conditions, and any use of any compounds identified using such a screening assay in such diseases or conditions.

Generally, high throughput screens for CI-MPR receptor modulators i.e. candidate or test compounds or agents (e.g., peptides, peptidomimetics, small molecules or other drugs) may be based on assays which measure biological activity of CI-MPR (or grB). The invention therefore provides a method (also referred to herein as a “screening assay”) for identifying modulators, which have a stimulatory or inhibitory effect on, for example, CI-MPR biological activity or expression, or which bind to or interact with CI-MPR proteins, or which have a stimulatory or inhibitory effect on, for example, the expression or activity of CI-MPR interacting proteins (targets) or substrates. In one embodiment, the screening assay is set-up to identify modulators of CI-MPR-grB interaction.

Examples of methods available for cell-based assays and instrumentation for screening including high-throughput screens for the biological activity of the present invention are shown hereinbelow. Numerous types of assays and screens are known in the art and can be adapted to the genes, proteins and biological activities of the present invention. Such assays include a determination of the expression level and externalization level of CI-MPR that can be measured in a cell line (recombinant or non-recombinant) using fluorescence-based assays, nucleic acid probes, FACS and the like. Other assays measure labeled grB or other molecules internalized by CI-MPR in cells (recombinant or non-recombinant).

In one embodiment, the invention provides assays for screening candidate or test compounds which interact with substrates of a CI-MPR protein or biologically active portion thereof.

In another embodiment, the invention provides assays for screening candidate or test compounds which bind to or modulate the activity of a CI-MPR protein or polypeptide or biologically active portion thereof. In a particular embodiment, such assays screen for compounds which modulate CI-MPR interaction with M6P-containing molecules (e.g. proteins, peptides or mimetics thereof).

In one embodiment, an assay is a cell-based assay in which a cell which expresses a CI-MPR protein or biologically active portion thereof, either natural or recombinant in origin, is contacted with a test compound and the ability of the test compound to modulate CI-MPR biological activity, e.g. binding and/or internalization of M6P-containing molecule [e.g. grB, grA, IGF-II], or binding to a M6P-containing molecule or a portion thereof, or any other measurable biological activity of CI-MPR is determined. Determining the ability of the test compound to modulate CI-MPR activity can be accomplished by monitoring, for example, an apoptotic marker such as DNA fragmentation, from a cell which expresses CI-MPR upon exposure of the test compound to the cell. Furthermore, determining the ability of the test compound to modulate CI-MPR activity can be accomplished by monitoring, for example, phosphatidylserine externalization, caspase 3 activation, from a cell which expresses CI-MPR upon exposure to a test compound. Assays used to monitor apoptotic markers in cells, CTL-killing and the like, can be adapted to identify modulators of CI-MPR function on these biological activities.

Determining the ability of the test compound to modulate binding of CI-MPR to a substrate (e.g. M6P-containing substrate) can be accomplished, for example, by coupling the CI-MPR-interacting agent or substrate with a radioisotope or enzymatic label such that binding of the CI-MPR substrate or agent to CI-MPR can be determined by detecting the labeled CI-MPR substrate in a complex. For example, compounds (e.g., CI-MPR agents or substrates) can be labeled with ¹²⁵1, ³⁵S, ¹⁴C, or ³H, either directly or indirectly, and the radioisotope detected by direct counting radio-emission or by scintillation counting. Alternatively, compounds can be enzymatically labeled with, for example, horseradish peroxidase or alkaline phosphatase. In these assays, compounds which inhibit or increase substrate binding to CI-MPR are useful for the therapeutic objectives of the invention.

It is also within the scope of this invention to determine the ability of a compound (e.g. CI-MPR substrate) to interact with CI-MPR without the labeling of any of the interactants.

In another embodiment, the assay is a cell-based assay comprising a contacting of a cell containing a target molecule or ligand (e.g. another molecule, substrate or protein that interacts with or binds to CI-MPR) with a test compound and determining the ability of the test compound to indirectly modulate (e.g. stimulate or inhibit) the biological activity of CI-MPR by binding or interacting with the target molecule. Determining the ability of the test compound to indirectly modulate the activity of CI-MPR can be accomplished, for example, by determining the ability of the test compound to bind to or interact with the target molecule and thereby to indirectly modulate CI-MPR, to modulate grB internalization, or to modulate other biological activities of CI-MPR. In a preferred embodiment, the cell-based assay comprises a contacting of a cell containing CI-MPR or functional parts thereof and an M6P-containing molecule (e.g. grB) with a test compound and determining the ability of the test compound to directly modulate (e.g. stimulate or inhibit) the biological activity of CI-MPR by modulating the interaction between CI-MPR or functional parts thereof and the M6P-containing molecule. Determining the ability of the CI-MPR protein or a biologically active fragment thereof, to bind to or interact with the target molecule or M6P-containing molecule can be accomplished by one of the methods described above or known in the art for determining direct binding. In one embodiment, determining the ability of the test compound's ability to bind to or interact with the target molecule or preferably to modulate the interaction between CI-MPR (or biologically active portion thereof and on M6P-containing molecule (preferably grB) and thereby to modulate the CI-MPR protein can be accomplished by determining a secondary activity of the target molecule or of an internalized M6P-containing protein or molecule (e.g. through IGF-II). For example, the activity of the target molecule can be determined by detecting induction of a cellular second messenger of the target, detecting catalytic/enzymatic activity of the target on an appropriate substrate, detecting the induction of a reporter gene or detecting a target-regulated cellular response. Alternatively, recombinant cell lines may employ recombinant reporter proteins which respond, either directly or indirectly to such secondary messengers, as known in the art.

In yet another embodiment, an assay of the present invention is a cell-free assay in which a CI-MPR protein or biologically active portion thereof, either naturally occurring or recombinant in origin, is contacted with a test compound and the ability of the test compound to bind to, or otherwise modulate the biological activity of, the CI-MPR protein or biologically active portion thereof is determined. Preferred biologically active portions of the CI-MPR proteins to be used in assays of the present invention include fragments which participate in interactions with M6P-containing molecules (e.g. M6P-containing proteins) or fragments thereof. Binding of the test compound to the CI-MPR protein can be determined either directly or indirectly as described above. In a preferred embodiment, the assay includes contacting the CI-MPR protein or biologically active portion thereof with a known compound which binds CI-MPR to form an assay mixture, contacting the assay mixture with a test compound, and determining the ability of the test compound to interact with a CI-MPR protein, wherein determining the ability of the test compound to interact with a CI-MPR protein comprises determining the ability of the test compound to preferentially bind to CI-MPR or biologically active portion thereof as compared to the known compound.

In another embodiment, the assay is a cell-free assay in which a CI-MPR protein or biologically active portion thereof is contacted with a test compound and the ability of the test compound to modulate (e.g., stimulate or inhibit) the activity of the CI-MPR protein or biologically active portion thereof is determined. Determining the ability of the test compound to modulate the activity of a CI-MPR protein can be accomplished, for example, by determining the ability of the CI-MPR protein to bind to a M6P-containing target molecule by one of the methods described above for determining direct binding. Determining the ability of the CI-MPR protein to bind to a M6P-containing target molecule can also be accomplished using a technology such as real-time Biomolecular Interaction Analysis (BIA, Sjolander, S. and Urbaniczky, C. (1991) Anal. Chem. 63:2338-2345 and Szabo et al. (1995) Curr. Opin. Struct. Biol. 5:699-705). As used herein, “BIA” refers to a technology for studying biospecific interactions in real time, without labeling any of the interactants (e.g. BIA core). Changes in the optical phenomenon of surface plasmon resonance (SPR) can be used as an indication of real-time reactions between biological molecules.

In an alternative embodiment, determining the ability of the test compound to modulate the activity of a CI-MPR protein can be accomplished by determining the ability of the test compound to modulate the activity of an upstream or downstream effector of a CI-MPR or M6P-containing target molecule. For example, the activity of the test compound on the effector molecule can be determined or the binding of the effector to CI-MPR can be determined as previously described.

The cell-free assays (as well as cell-based, as exemplified herein) of the present invention are amenable to use of both soluble and/or membrane-bound forms of isolated proteins. In the case of cell-free assays in which a membrane-bound form of an isolated protein is used, it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton®X-114, Thesit®, Isotridecypoly(ethylene glycol ether)n. 3-[(3-cholamidopropyl)dimethy-amino]-I-propane sulfonate (CHAPS), 3-[(3-cholamidopropyl)dimethylamino]-2-hydroxy-I-propane sulfonate (CHAPSO), or N-dodecyl-N,N-dimethyl-3-ammnonio-I-propane sulfonate.

In more than one embodiment of the above assay methods of the present invention, it may be desirable to immobilize either CI-MPR or its target molecule or ligand to facilitate separation of complexed from uncomplexed forms of one or both of the proteins, as well as to accommodate automation of the assay. Binding of a test compound to a CI-MPR protein or interaction of a CI-MPR protein with a target molecule or ligand in the presence and absence of a candidate compound, can be accomplished in any vessel suitable for containing the reactants. Examples of such vessels include microtitre plates, test tubes and micro-centrifuge tubes. In one embodiment a fusion protein can be provided which adds a domain that allows one or both of the proteins to be bound to a matrix. For example. glutathione-S-transferase/CI-MPR fusion proteins or glutathione-S-transferase/target fusion proteins (e.g. glutathione-S-transferase/grB proteins) can be adsorbed onto glutathione sepharose beads (Sigma Chemical, St. Louis, Mo.) or glutathione derivatized microtitre plates, which are then combined with the test compound or the test compound and either the non-adsorbed target protein or CI-MPR protein and the mixture incubated under conditions conducive to complex formation (e.g. at physiological conditions for salt and pH). Following incubation the beads or microtitre plate wells are washed to remove any unbound components, the matrix immobilized in the case of beads, complex determined either directly or indirectly, for example, as described above. Alternatively, the complexes can be dissociated from the matrix, and the level of CI-MPR binding or activity determined using standard techniques.

Other techniques for immobilizing proteins on matrices (and well-known in the art) can also be used in the screening assays of the invention. For example, either a CI-MPR protein or a CI-MPR target molecule or ligand can be immobilized utilizing conjugation of biotin and streptavidin. Biotinylated CI-MPR protein or target molecules or ligand can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with CI-MPR protein, target molecules or ligand but which do not interfere with binding of the CI-MPR protein to its target molecule or ligand can be derivatized to the wells of the plate, and unbound target or CI-MPR protein trapped in the wells by antibody conjugation. Methods for detecting such complexes, in addition to those described above for the GST-immobilized complexes, include immunodetection of complexes using antibodies reactive with the CI-MPR protein or target molecule, as well as enzyme-linked assays which rely on detecting an enzymatic activity associated with the CI-MPR protein or target molecule and in particular with grB.

In a preferred embodiment, candidate or test compounds or agents are tested for their ability to inhibit or stimulate a CI-MPR molecule's ability to modulate M6P-containing protein transport in a cell (such assays are described in for example Komada et al. 1999, Genes Dev.13(11):1475-85, and Roth et al. 1999, Chem. Phys. Lipids. 98(12):141-52).

In another embodiment candidate, or test compounds or agents are tested for their ability to inhibit or stimulate or regulate the phosphorylation state of a CI-MPR protein or portion thereof, or an upstream or downstream target protein, and more particularly a M6P-containing protein and especially grB using for example an in vitro kinase assay.

In another preferred embodiment, candidate or test compounds or agents are tested for their ability to inhibit or stimulate a CI-MPR molecule's ability to associate with (e.g. bind) mannose-6-phosphatase or multivalent M6P-containing molecules.

In another embodiment, modulators of CI-MPR expression are identified in a method wherein a cell is contacted with a candidate compound and the expression of CI-MPR mRNA or protein in the cell is determined. The level of expression of CI-MPR mRNA or protein in the presence of the candidate compound is compared to the level of expression of CI-MPR mRNA or protein in the absence of the candidate compound. The candidate compound can then be identified as a modulator of CI-MPR expression based on this comparison. For example, when expression of CI-MPR mRNA or protein is greater (statistically significantly greater) in the presence of the candidate compound than in its absence, the candidate compound is identified as a stimulator of CI-MPR mRNA or protein expression. The level of CI-MPR mRNA or protein expression in the cells can be determined by methods described herein or other methods known in the art for detecting CI-MPR mRNA or protein. In view of the importance of the externalized localization of CI-MPR in effecting internalization of grB, a determination of the cellular localization of CI-MPR in the cell is preferable. In another embodiment, modulators of CI-MPR traficking are identified by comparing the cellular localization of CI-MPR in the cell (e.g. at the surface thereof) in the presence versus in the absence of a candidate compound.

The assays described above may be used as initial or primary screens to detect promising lead compounds for further development. Often, lead compounds will be further assessed in additional, different screens. Therefore, this invention also includes secondary CI-MPR screens which may involve biological assays utilizing animal or mammalian cell lines expressing CI-MPR.

Tertiary screens may involve the study of graft rejection as exemplified herein. Accordingly, it is within the scope of this invention to further use an agent identified as described herein in an appropriate animal model. For example, an test compound identified as described herein (e.g., a CI-MPR modulating agent, an antisense CI-MPR nucleic acid molecule or conversaly anti-CI-MPR nucleic acid molecule, a CI-MPR-specific antibody, or a CI-MPR-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment (e.g. cancer, auto-immune diseases, viral infections and other diseases associated with a deregulation of grB internalization in cells), as described herein.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. ( 1994), J. Med. Chem. 37:2678; Cho et al. (1993) Science 261:1303; Carrell et al. (1994) Angew. Chem, Int. Ed Engl. 33:2059; Carell et al. (1994) Angew. Chem. Jnl. Ed. Engl. 33:2061; and in Gallop et al. (1994). Med Chem. 37:1233. Libraries of compounds may be presented in solution (e.g. Houghten (1992) Biotechniques 13:412-421). or on beads (Lam (199]) Nature 354:82-84), chips (Fodor (1993) Nature 364:555-556). bacteria (Ladner U.S. Pat. No. 5,223,409), spores (Ladner U.S. Pat. No. '409), plasmids (Cull et al.(1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) Proc. Natl. Acad. Sci. USA. 90:6909; Erb et al. (1994) Proc. Natl. Acad .Sci. USA 91: 11422; Zuckermann et al. (1994),.J: Med. Chem. 37:2678; Cho et al. (1993), Science 261:1303; Carrel1 et al. (1994) Angew. Chem Int. Ed. Engl. 33:2059, or luciferase, and the enzymatic label detected by determination of conversion of an appropriate substrate to product.

In summary, based on the disclosure herein, those skilled in the art can develop CI-MPR screening assays which are useful for identifying compounds which are useful for treating or preventing diseases, disorders or conditions associated with a deregulation or abnormal internalization of granzyme B in cells. The assays of this invention may be developed for low-throughput, high-throughput, or ultra-high throughput screening formats.

The assays of this invention employ either natural or recombinant CI-MPR protein (or grB protein and other ligands thereof or of CI-MPR). Cell fraction or cell free screening assays for modulators of CI-MPR biological activity can use in situ, purified, or purified recombinant CI-MPR proteins. Cell based assays can employ cells which express CI-MPR protein naturally, or which contain recombinant CI-MPR gene constructs, which constructs may optionally include inducible promoter sequences. In all cases, the biological activity of CI-MPR can be directly or indirectly measured; thus modulators of CI-MPR biological activity can be identified. The modulators themselves may be further modified by standard combinatorial chemistry techniques to provide improved analogs of the originally identified compounds.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, the DNA construct can be administered directly to the afflicted individual, for example, by injection in the bone marrow. The DNA construct can also be delivered through a vehicle such as a liposome or viral vector (e.g. adenoviral vector), which can be designed to be targeted to a specific cell type, and engineered to be administered through different routes. In one particular embodiment, the DNA construct would enable expression of CI-MPR to enable an increased internalization of grB.

For administration to humans, the prescribing medical professional will ultimately determine the appropriate form and dosage for a given patient, and this can be expected to vary according to the chosen therapeutic regimen (e.g. DNA construct, protein, cells), the response and condition of the patient as well as the severity of the disease.

Composition within the scope of the present invention should contain the active agent (e.g. fusion protein, nucleic acid, hormone, growth factor, soluble receptor and molecule) in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

While the term “individual” refers preferably to humans, it is used herein broadly to relate to animals in general.

The present invention relates to a kit for diagnosing a disease or condition or a predisposition to contracting same wherein the disease or condition is associated with a defect in the activity and/or the level of CI-MPR, and/or with a defect in internalization of grB, comprising a nucleic acid, a protein or a ligand in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (DNA protein or cells), a container which contains the primers used in the assay, containers which contain enzymes, containers which contain wash reagents, and containers which contain the reagents used to detect the extension products.

The present invention also relates to a kit comprising the oligonucleotide primer of the present invention, which are specific to CI-MPR, and/or CD-MPR, and/or grB.

The present invention is illustrated in further detail by the following non-limiting examples.

### EXAMPLE 1

### Dissection of the grB-dependent Apoptosis Pathway in Cells and Identification of the Receptor Responsible for grB Internalization

Cell Lines and Reagents

Murine L cells were grown in Dulbecco's modified Eagle's medium (DMEM, Gibco) supplemented with 10% FBS and with 2 mM L-glutamine and 100 μg/ml penicillin/streptomycin. The CD-MPR⁺ (SR2-1) and CI-MPR⁺ (MS9-II) cells were derived by transfecting human CD- or CI-MPR into an L cell line deficient in endogenous CI-MPR (L(Rec⁻)), while the MPR⁻ (MS) line was derived by transfection of L(Rec⁻) cells with vector alone (Gabel et al., 1983; Watanabe et al., 1990) and maintained as previously described (Watanabe et al., 1990). Human CTL and Jurkat cells were isolated and maintained as previously described (Atkinson et al., 1998). Human replication-deficient AD type 5 d170-3 and ADLacZ have been described elsewhere (Bett et al., 1994; Addison et al., 1997). Rabbit antisera with specificity for the human CI-MPR (Wood et al., 1991) or CD-MPR (Ma et al., 1992) have been described previously. Goat anti-rabbit-FITC was obtained from Jackson lmmunoResearch and streptavidin-cychrome from PharMingen. Murine specific CD4 (L3T4) and CD8 (53-6.7) mAb were obtained from Cedarlane. Secondary biotinylated Ab for immunohistochemical staining included goat anti-mouse IgG and goat anti-rat IgG obtained from Cedarlane and Vector, respectively. Anti-Fas (CD95) mAb specific to mouse (Jo2) or human (APO-1) was obtained from PharMingen. Human sCI-MPR has been described (Valenzano et al., 1995). M6P and other related monosaccharides, as well as calf-intestinal phosphatase were purchased from Sigma. The catalytic subunit of protein phosphatase-1 has been described (Bagu et al, 1997).

Purification of GrB and Preparation of Labeled GrB Probe

Human grB was purified from the cytolytic granules of YT-Indy cells as described previously (Caputo et al., 1999). GrB was derivatized with sulfo-NHS-biotin (Pierce) or OG 488 (Molecular Probes) as per the manufacturer's directions for labeling protein. Derivatized grB was separated from unbound probe by passing the material through a Sephadex G-25 column (HiTrapθ Desalting column, Pharmacia Biotech) with saline or phosphate-buffered saline (PBS) as the buffer. Typically the concentration of unlabeled grB was ˜50 μg/ml, whereas the estimated concentration of derivatized material was ˜5 μg/ml. GrB was assayed for enzymatic activity using an ASPase assay as previously described (Caputo et al., 1993).

Flow Cytometric Analysis of Cell Surface MPR Expression

Cells (2×10⁵) were incubated for 20 min at 4° C. with a 200-fold dilution of rabbit anti-CI- or CD-MPR and washed. Cells were then incubated with a 200-fold dilution of goat-anti-rabbit-FITC, washed, and analyzed with a Becton Dickenson FACScanθ equipped with CELLQuestθ software. PBS/0.1% BSA buffer was used throughout the procedure. For all flow cytometric analysis, over 10,000 events (typically 15,000) were recorded. Results are presented as the relative mean fluorescence intensity (MFI), which represents the MFI of the population labeled with anti-MPR antisera and secondary Ab minus the MFI obtained with secondary Ab treatment alone.

GrB Binding Assay

Cells (1-2×10⁵) were washed twice and resuspended in buffer before the addition of grB-biotin or -OG in a final volume of 20 μl for a 30-60 min incubation and washed again twice. In the case of grB-biotin, cells were then incubated with streptavidin-cychrome for 20 min and washed twice. Unless otherwise indicated, ˜25 ng grB-biotin or -OG was used to label cells. All steps of the assay were carried out at 4° C. to prevent intracellular uptake of grB. PBS/0.1% BSA buffer was used throughout the procedure. In some cases, results are presented as the relative MFI, which represents the MFI of the grB-labeled population minus the MFI of the unlabeled population.

Phosphatase Treatment of GrB

GrB was treated for 1 hr at 37° C. in a phosphate-free (saline) or a high-phosphate (64.5 mM Na₂HPO₄.7H₂O, 21 mM KH₂PO₄) buffer with calf intestinal alkaline phosphatase (1-10 U), 20 nM of the 37 kDa catalytic subunit of recombinant protein phosphatase-1 (Bagu et al, 1997), or with vehicle control. Cells were then incubated with the treated grB at 4° C. for 30-60 min. For the TUNEL or annexin V assays, cells were then washed twice before incubation at 37° C. with AD.

M6P Inhibition Studies

M6P and various other monosaccharides at different concentrations were added to cells for 15 min at 4° C. before the addition of labeled grB, or at room temperature before the addition of grB and AD.

GrB Uptake Assay

Cells on coverslips were incubated for 20 min at 37° C. with DMEM/0.1% BSA containing 25 ng grB-OG that was untreated, mock-treated, or alkaline phosphatase-treated as described above. For the M6P inhibition studies, cells were treated with 20 mM M6P in DMEM/0.1% BSA for 10 min at 37° C., before the addition of grB-OG, as above. Cells were then washed twice with PBS/0.1% BSA, fixed with 2% paraformaldehyde in PBS, and allowed to dry briefly before the coverslips were mounted and examined by confocal microscopy. Imaging was performed with a Zeiss Axiovertθ 100M LSM510 laser scan microscope and analyzed using LSM510 software.

sCI-MPR Inhibition Studies

GrB was incubated with sCI-MPR at various concentrations in PBS/0.1% BSA for 30 min at 4° C. Subsequently, cells were incubated with grB/sCI-MPR for 30-60 min at 4° C. and washed twice, before analysis of binding, or before the addition of AD for TUNEL and annexin V analysis.

TUNEL and Annexin V Apoptosis Assays

For the in vitro killing assays, 10 pfu per cell of replication-deficient adenovirus (AD) were added subsequent to grB addition. Analysis of DNA fragmentation by TUNEL analysis has been described previously (Heibein et al., 1999). TUNEL materials (Roche Diagnostics) were used as per the manufacturer's instructions. Phosphatidylserine externalization was measured as previously described (Heibein et al., 1999) using annexin V-FITC (PharMingen). Percent specific TUNEL/annexin V positive cells was calculated as: [(% positive labeling cells with grB and AD-% positive labeling cells without grB and AD)/(100-% positive labeling cells without grB and AD)]×100.

In vivo Killing Assays

The ⁵¹Cr- (lysis) and ³H-thymidine (DNA fragmentation) release assays have been previously described (Darmon et al., 1996). Target cells (1×10⁴ cells/well for the ⁵¹Cr-release assay and 5×10⁴ cells/well for the ³H-thymidine-release assay) were incubated with CTL at various E:T ratios (effector:target) in the presence or absence of blocking murine- (1 μg/ml) or human- (200 ng/ml) anti-Fas mAb. Data from one experiment represent the mean of data from triplicate samples. Typically, spontaneous ⁵¹Cr-release and DNA fragmentation was less than 10% and 5%, respectively. Percent specific lysis and DNA fragmentation was calculated as: [(sample cpm−spontaneous cpm)/(total cpm−spontaneous cpm)]×100.

Transplantation and Graft Characterization

MPR⁻ or CI-MPR⁺ cells were transplanted under the left kidney capsule of anesthetized SCID-beige or BALB/c mice. Aliquots consisting of 2×10⁶ cells were aspirated into polyethylene tubing, pelleted, and gently placed under the kidney capsule with the aid of a micromanipulator syringe. Once the tubing was removed, the capsulotomy was cauterized. At 7- and 14-days post-transplantation, grafts were excised from surrounding kidney tissue, embedded, and stored at −80° C. Frozen 5 μm tissue sections were fixed with acetone and stained for 30 min with the H-2^{k}-specific mAb 11-4.1 or with CD4- or CD8-specific mAb. After labeling with the appropriate secondary biotinylated Ab, an avidin-biotin complex/horseradish peroxidase method was used and developed with 3,3-diaminobenzidinetetrahydrochloride. Positive controls included frozen sections of CBAJ mouse liver for H-2^{k} detection and BALB/c mouse spleens for CD4 and CD8 detection. Negative controls, which resulted in negative staining consisted of omission of the primary mAb. The experiments were conducted such that the identity of the donor cells was coded and only revealed after all data had been analyzed.

Phosphorylation of grB is Required for Cell Surface Binding and Apoptosis Induction

In our search to characterize a receptor for grB, we developed a binding assay using purified human grB conjugated to biotin or the fluorescent dye Oregon Green 488 (OG) as a probe. Labeled grB retained enzymatic activity and bioactivity. Binding was assessed by flow cytometry after incubation of cells with labeled grB at 4° C. and both grB-OG and grB-biotin bound to Jurkat cells in a manner that was dose-dependent, saturable and that was inhibitable with unlabeled grB. Using labeled grB, we tested the ability of a number of factors and conditions to modulate this binding. Our approach was guided by the surprising finding that recombinant grB was not as biologically active as purified granule-derived grB, although they were comparable in enzymatic activity (data not shown). One possibility that could explain this discrepancy would be a difference in the post-translational processing of grB.

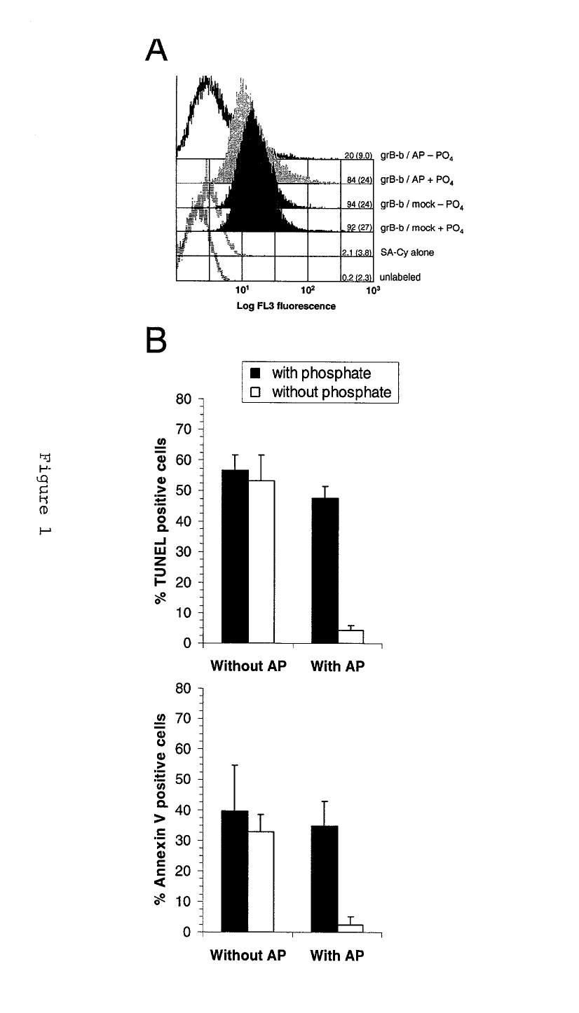

In order to test the importance of phosphorylation Jurkat cells were incubated at 4° C. with grB-biotin that had been treated at 37° C. with alkaline phosphatase in phosphate-free buffer. As a control, grB was treated without phosphatase, or with phosphatase in a phosphate-containing buffer to inhibit phosphatase activity (Lakshmi and Balasubramanian, 1980). Treatment of grB with phosphatase in a phosphate-free buffer had no effect on the enzymatic activity of grB, but did result in a marked reduction in its ability to bind Jurkat cells (FIG. 1A).

If the internalization of grB by receptor-mediated endocytosis is necessary for apoptosis, then grB that does not bind to the cell surface should be ineffective in killing (i.e. in triggering the apoptotic pathway). To test this, Jurkat cells were incubated for 3 hr with phosphatase-treated grB and AD and analyzed using a TUNEL or annexin V assay. Less than 5% of cells were TUNEL positive after incubation with dephosphorylated grB (FIG. 1B) compared with 50% TUNEL positive cells after treatment with grB pretreated with alkaline phosphatase in phosphate-containing buffer. Similar results were observed using annexin V as a marker for apoptotic cells (FIG. 1B). In order to distinguish whether these results were due to the dephosphorylation of carbohydrate or of amino acid moieties, grB was treated with the 37 kDa catalytic subunit of recombinant protein phosphatase-1 (a serine/threonine/tyrosine phosphatase) under conditions where other proteins are effectively dephosphorylated (Bagu et al, 1997). We found that grB did not lose enzymatic activity or its ability to bind and to induce apoptosis of Jurkat cells (data not shown), and thus we conclude that phosphorylation of carbohydrate is important.

GrB Binds to the MPR to Induce Apoptosis

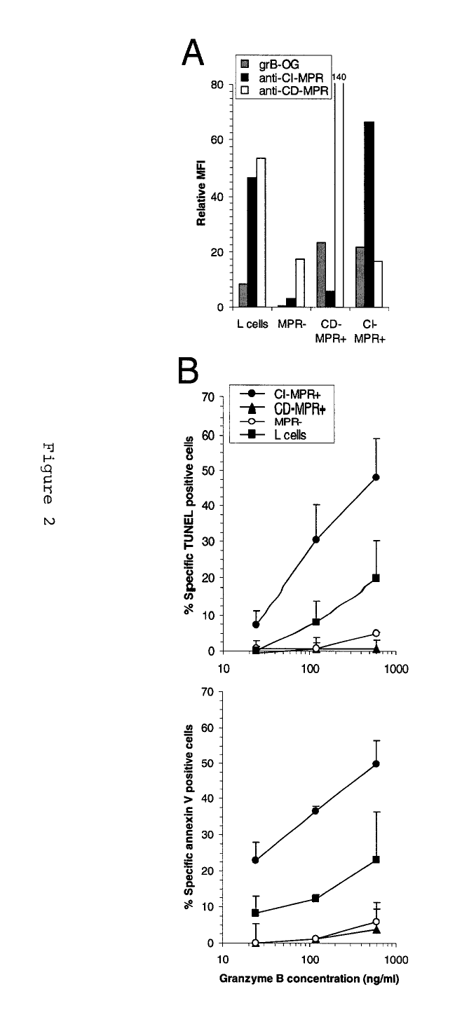

Two types of mannose 6-phosphate receptors (MPR) have been described, the ˜270 kDa cation-independent MPR (CI-MPR) also known as the insulin-like growth factor receptor (IGF-IIR), and the 46 kDa cation-dependent MPR (CD-MPR) (reviewed in Kornfeld, 1992; Dahms, 1996; Munier-Lehmann et al., 1996). Both are type I glycoproteins that have a high binding affinity for lysosomal enzymes and other proteins that contain phosphomannosyl residues. Using wild-type L cells and L cells that were either deficient in the expression of CI-MPR (MPR⁻ cells), or that were transfected to overexpress the CI-MPR (CI-MPR⁺ cells) or the CD-MPR (CD-MPR⁺ cells) (Gabel et al., 1983; Watanabe et al., 1990), we found a direct correlation between cell surface MPR expression and grB binding (FIG. 2A). In addition, in agreement with the results obtained with Jurkat cells, treatment of grB with alkaline phosphatase in the absence of phosphate resulted in a dramatic decrease in binding to L cells and the MPR-transfected L cell lines. Furthermore, by confocal analysis of CI-MPR⁺ cells after incubation with grB-OG at 37° C., we observed intracellular localization of grB that was treated with alkaline phosphatase in the presence of phosphate, but not with grB that was dephosphorylated (data not shown).

The L cell series was then tested for susceptibility to killing by grB and AD. The MPR⁻ line was relatively resistant to grB and AD-induced DNA fragmentation (FIG. 2B). Conversely, the CI-MPR⁺ and wild-type L cells underwent DNA fragmentation and bound annexin V after treatment (FIG. 2B). At the time-point of the assay about 30% of the CI-MPR⁺ and L cells were TUNEL and annexin V positive, however at later times this number approached 100% (data not shown). In stark contrast the CD-MPR⁺ line was resistant to grB and AD-mediated DNA fragmentation and did not bind annexin V (FIG. 2B).

Inhibition of GrB Binding and Uptake by M6P

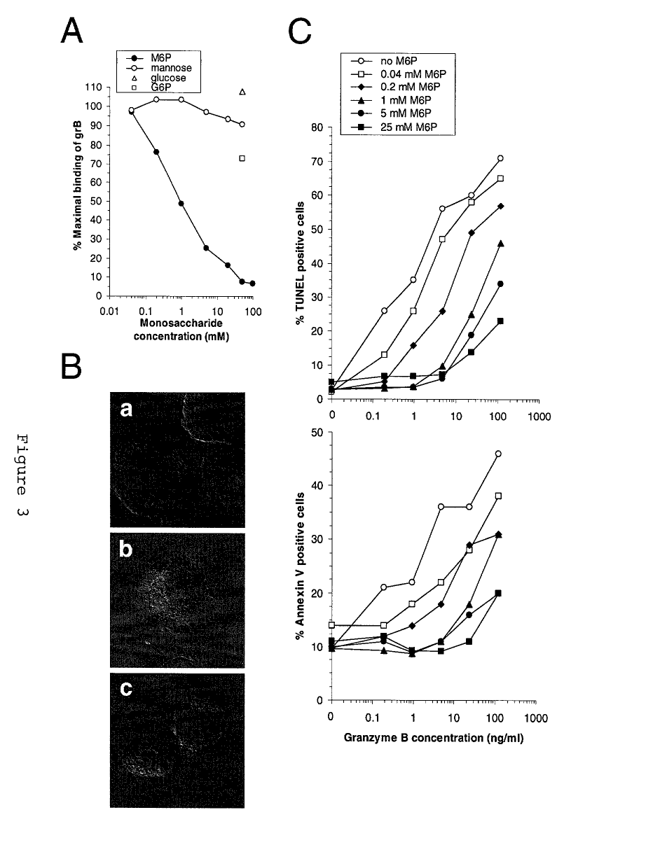

The binding and uptake of acid hydrolases by the MPR is blocked by mannose 6-phosphate (M6P) (Kaplan et al., 1977). Cells were treated at 4° C. with various concentrations of M6P together with labeled grB and analyzed by flow cytometry. At a concentration of 20 mM, M6P considerably reduced binding of grB to Jurkat cells with half-maximal inhibition occurring at a concentration of 1 mM M6P (FIG. 3A). Mannose and glucose had no effect on grB binding, whereas glucose 6-phosphate had a slight inhibitory effect that was only evident at concentrations above 25 mM (FIG. 3A). Similar results were observed with the wild-type L cells, or with the CI-MPR⁺ and CD-MPR⁺ cells (data not shown).

Finally, the effect of M6P on the uptake of grB was assessed. CI-MPR⁺ cells were incubated at 37° C. with grB-OG with or without 20 mM M6P and analyzed by confocal microscopy. In the presence of M6P essentially no grB could be detected within the cells (FIG. 3B).

Inhibition of GrB/AD-Mediated Apoptosis by M6P

A critical question was whether the grB-MPR interaction at the cell surface correlated with grB biological activity. To examine this, Jurkat cells were treated with or without M6P or other sugars along with grB and AD for 3 hr, and analyzed for apoptosis (FIG. 3C). Significantly, we found that 200 μM or greater of M6P markedly reduced in a dose-dependent manner the percentage of TUNEL or annexin V positive cells (FIG. 3C). In contrast, treatment with other related sugars or sugar phosphates had little effect on grB-mediated apoptosis (FIG. 3C). Similar results were seen with L cells and CI-MPR⁺ cells. No effect of M6P was observed on infection of AD containing a β-galactosidase insert. Finally, using Jurkat cells, we found that the preincubation of grB with soluble CI-MPR (≧100 μg/ml) markedly reduced in a dose-dependent manner both the cell surface binding of grB, and grB/AD-mediated apoptosis (data not shown). The affinity of M6P-proteins to the CD-MPR has been found to be about 100 times less than to the CI-MPR. It is expected that a soluble CD-MPR, while less efficient than CI-MPR, would be more efficient in blocking grB uptake. CD-MPR or fragments thereof could be engineered to become more efficient blockers of grB uptake.

Correlation of CTL-Mediated Target Cell Apoptosis with Cell Surface CI-MPR Expression

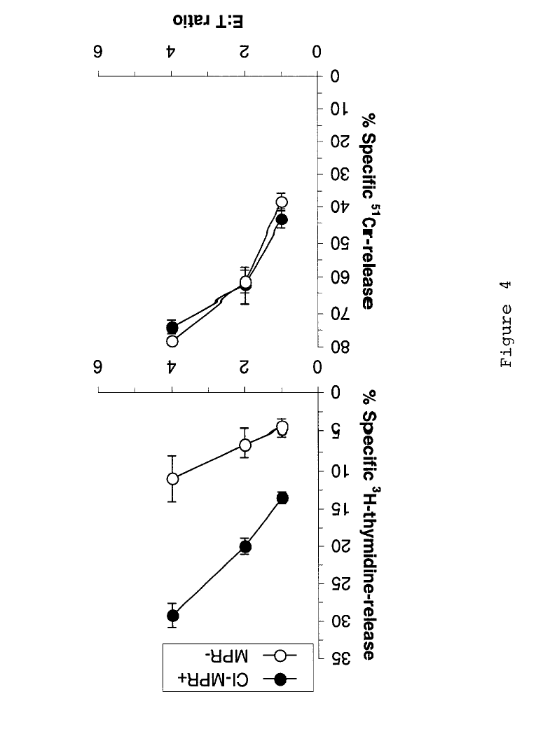

Taken together, the results presented herein implicate an essential role for the CI-MPR in the binding and uptake of grB in vitro. However, it was critical to determine the importance of the CI-MPR in CTL-mediated killing, and thus we used MPR⁻ and CI-MPR⁺ cells as targets with a CTL line at various effector:target ratios. The extent of target cell DNA fragmentation was assessed by ³H-thymidine release after 3 hr and membrane damage by ⁵¹Cr-release after 5 hr. CTL-mediated apoptotic death of targets within the first 3 hr is mediated by grB whereas granzyme A (grA)- and Fas-mediated killing are detected at later times (Shresta et al., 1998). Incubation of effectors and targets in the presence of a blocking anti-Fas monoclonal antibody (mAb) had no effect on DNA fragmentation or ⁵¹Cr-release, indicating that killing within this time period was Fas-independent. This mAb abrogated DNA fragmentation of these target cells by a perforin-deficient murine CTL line after a 4-hr incubation (data not shown). After incubation with CTL, the MPR⁻ target cells showed a low amount of DNA fragmentation but a significant amount of ⁵¹Cr-release which increased with the effector:target (E:T) cell ratio (FIG. 4). However, in comparison there was much greater DNA fragmentation with CI-MPR⁺ target cells, although the extent of ⁵¹Cr-release was similar to that of MPR⁻ cells (FIG. 4).

We attempted to assess the importance of the MPR in CTL-mediated killing by incubating the effectors with targets in the presence of M6P. However, half-maximal inhibition of target cell DNA fragmentation was only evident with M6P at unphysiological concentrations (>25 mM) (data not shown). Thus, it appears that in order to be a more efficient inhibitor (or interacting factor to CI-MPR) of grB binding/internalization by CI-MPR that the agent or compound (e.g. ligand) tested must contain more than one M6P binding site. Since grB contains two M6P binding sites, it appears that preferably, the agent should contain at least two M6P binding sites.

Transplant Rejection Proceeds Through the CI-MPR

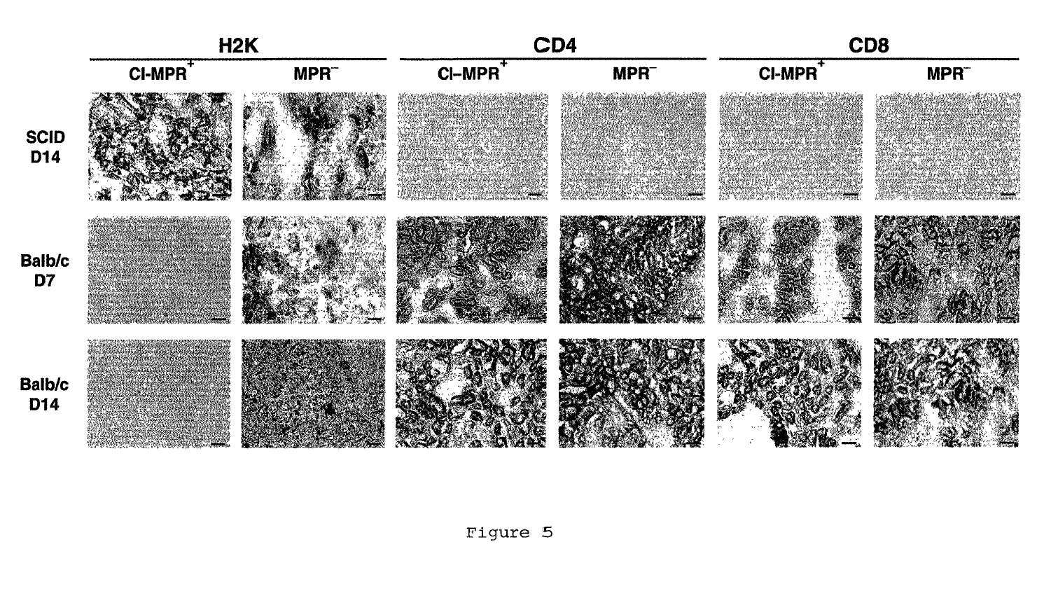

The in vivo significance of the results presented above was evaluated using transplantation of allogeneic cells. Cells were transplanted under the kidney capsule since this has been shown to be a suitable anatomical space to maintain the survival of cellular grafts such as pancreatic islets (Diamond and Gill, 2000) and hepatocytes (Ohashi et al., 2000). Furthermore, since this site maintains the cells in close proximity to each other, it is also useful for determining the degree of graft rejection as well as assessing the phenotypes of cellular infiltrates. By microinjection, donor H-2^{k}-expressing MPR⁻ or CI-MPR⁺ cells were transplanted under the kidney capsule of BALB/c-recipients (H-2^{d}). Graft cell survival was monitored after 7 and 14 days by immunostaining kidney capsule tissue with mAb specific for H-2^{k}, CD4, and CD8. As a control, donor cells were also transplanted in SCID hosts and monitored in the same way. The results of the transplant into BALB/c recipients were clear: at day 7 the CI-MPR-expressing donor cells were completely rejected, whereas at day 14 CI-MPR-deficient donor cells remained in the kidney capsule where they appeared to have expanded (FIG. 5). In the lymphocyte-deficient SCID host, both MPR⁻ and CI-MPR⁺ cells survived the engraftment (FIG. 5). CD4⁺ and CD8⁺ cells were present in the BALB/c kidney capsule indicating lymphocyte infiltration with both cell lines. In the case of CI-MPR⁺ cells, this appeared to lead to rejection, but the MPR⁻ cells survived despite the significant infiltrate. Parental wild-type L cells were also rejected with similar kinetics (data not shown).

Discussion

GrB Binding and Uptake by the Cell Surface MPR

The CI- and CD-MPR are found predominantly inside the cell where they play key roles in targeting M6P-expressing proteins from the Golgi to a pre-lysosomal compartment (Griffiths et al., 1990). Both receptors are also present at the cell surface, although the CI-MPR is thought to be the predominant receptor for the binding and uptake of M6P-containing molecules (Stein et al., 1987; Ma et al., 1991). We found that granule-purified grB bound at negligible levels to cells deficient in CI-MPR expression, but at high levels to CI-MPR deficient cells transfected to overexpress the CI-MPR or the CD-MPR (FIG. 2A). The dephosphorylation of grB markedly prevented its binding (FIG. 1A) and uptake (data not shown), as did coincubation with M6P (FIG. 3A) or soluble CI-MPR (data not shown). Taken together, these findings support a model whereby grB binding and uptake involves an interaction with cell surface MPR that is dependent on the expression of the M6P recognition marker on grB.

The CI-MPR and CD-MPR are usually thought of as being important in the trafficking of acid hydrolases to lysosomes (Nolan and Sly, 1987). Griffiths and Isaaz (1993) demonstrated that grA is phosphorylated on high-mannose residues, and that this modification is important in the intracellular targeting of the granzyme to the cytolytic granules. It was also suggested that grB may be similarly phosphorylated (Griffiths and Isaaz, 1993). Most lysosomal proteins that contain the M6P signal are rapidly dephosphorylated once they reach the lysosome (Einstein and Gabel, 1991), and it might be presumed that M6P-containing granzyme would also lose its phosphates within the cytolytic granules, which have been described as secretory lysosomes (Page et al., 1998). Based on our findings, we suggest that grB possesses the M6P modification and that this modification is retained in the cytolytic granule. Retention of the M6P modification may be explained by the observation that grB is located in the granule core whereas phosphatase is present in the granule cortex (Page et al., 1998). Neutrophil azurphilic granules, which are similar to cytolytic granules, also contain mannose 6-phosphorylated proteins (Cieutat et al., 1998).

At the cell surface the CI-MPR is constitutively endocytosed, where its main role is thought to be the binding and internalization of the non-glycosylated polypeptide hormone IGF-II (Oka and Czech, 1986), with a minor role being the reuptake of secreted acid hydrolases (Griffiths et al., 1988; Dahms, 1996). We now suggest that a critical function of the CI-MPR is the binding and uptake of grB. Other non-acid hydrolase proteins besides IGF-II that bind the cell surface CI-MPR include the precursor form of transforming growth factor-β (pTGF-β) (Kovacina et al., 1989), leukemia inhibitory factor (Blanchard et al., 1998), and the Herpes simplex virus (Brunetti et al., 1994).

Interaction of GrB with the CI-MPR is Critical for Apoptosis

The extent of grB-mediated apoptosis was much greater for CI-MPR⁺ cells compared with MPR⁻ cells (FIG. 2B), and apoptosis was inhibited by the coincubation of grB with M6P (FIG. 3C), soluble CI-MPR (sCI-MPR) (data not shown), or by the prior treatment of grB with phosphatase (FIG. 1B). Importantly, there was a direct correlation between binding and uptake of grB and apoptosis (FIGS. 3A, 3B and 3C). That some apoptosis was evident for MPR⁻ cells at high concentrations of grB may be explained by the very low-level (˜10% of wild-type) of endogenous CI-MPR present (Lobel et al., 1989) or alternatively to limited entry of grB in a non-MPR fashion.

It is interesting that recombinant forms of grB that would not be mannose 6-phosphorylated do induce apoptosis in vitro (Beresford et al., 1999; Shresta et al., 1999). However, this grB was only effective in killing at high concentrations. In one study, 5-20 μg/ml of yeast-derived recombinant grB was required to induce apoptosis, and this was estimated to be approximately 5-20-fold more than the amount of grB that would be found in all the effector cells in a typical in vitro CTL assay (Shresta et al., 1999). In comparison we typically used granule-purified grB at concentrations of ˜50 ng/ml, and less than 1 ng/ml was sufficient to elicit effective Jurkat cell killing (FIG. 3C). We cannot rule out a minor MPR-independent pathway, and this may explain the DNA fragmentation seen with CTL acting on MPR⁻ cells at high E:T ratios. Perhaps a high local concentration of grB can be achieved at the CTL-target interface such that some uptake can occur independently of the MPR. However, it is clear that this effect is not relevant to the in vivo rejection model (FIG. 5).

The inability of overexpressed CD-MPR to induce grB-mediated apoptosis (FIG. 2B) may be due to the inefficiency of the CD-MPR to endocytose bound ligand (Munier-Lehmann et al., 1996). Indeed we have observed essentially no grB uptake in the CD-MPR-overexpressing CD-MPR⁺ cells (BM and RCB, unpublished findings).

Expression of the CI-MPR is Critical for CTL-Mediated Target Cell Apoptosis

In agreement with our model of CI-MPR mediated uptake of grB, we found that a CTL line efficiently induced apoptosis in target cells only when the targets expressed transfected CI-MPR (FIG. 4). Similar findings were obtained with other CTL effectors including activated primary splenocytes (BM and RCB, unpublished findings). Interestingly, it was the DNA fragmentation component that was effectively prevented in CI-MPR-deficient targets and not the disruption of the plasma membrane. The most likely explanation for this is that membrane damage is being induced by perforin in the absence of efficient binding and uptake of grB. Likewise, the absence of early DNA fragmentation with later ⁵¹Cr-release has been documented in killing assays that used grB-deficient CTL (Heusel et al., 1994).

The membrane damage observed in cell culture assays is attributed to perforin but the relevance of this to in vivo cytotoxicity has recently been questioned (Shresta et al., 1999). Therefore, we sought to address the importance of grB binding and uptake by the CI-MPR using an in vivo model. We observed that the expression of CI-MPR was critical for the clearance of transplanted allogeneic cells. Thus cells that were deficient in the receptor survived despite the infiltration of both CD4 and CD8 T cells (FIG. 5). In contrast, cells transfected to overexpress the receptor (FIG. 5) or wild-type L cells (data not shown) were efficiently rejected. Our in vivo data differed from our in vitro findings in that in the transplantation study the MPR⁻ cells survived (FIG. 5), whereas there was significant ⁵¹Cr-release/lysis of MPR⁻ cells in vitro (FIG. 4). This underscores the questionable relevance of the perforin-mediated ⁵¹Cr-release assay compared with what occurs in vivo. Differences in the effective E:T ratios may at least partially explain the differences between killing in vivo and in vitro. Our in vitro data demonstrates the importance of receptor expression for binding and uptake of grB but the in vivo result underlines that expression of CI-MPR is paramount for cell death.

In addition to the importance of these studies to define the mechanism of granule-mediated cytotoxicity, the results are also significant for understanding graft rejection. There has been considerable debate as to the relevance of antibodies, cytokines, T cell subsets, and macrophages to the rejection process. Undoubtedly these play an important role but our data strongly suggests that the granzyme pathway used by CTL is paramount at the effector stage. The CI-MPR⁻ cells transplanted under the kidney were surrounded by CD4⁺ and CD8⁺ T cells and are presumably awash in cytokines and antibodies. In addition, it is likely that activated macrophages and their lytic proteins, and Fas ligand positive cells were present. Despite all this, in the absence of the grB receptor, the allogeneic cells survived.

As to the importance of other granzymes, it has recently been shown that grB supplemented by grA is important for graft-versus-host disease after bone marrow transplantation (Shresta et al., 1999). That the CI-MPR⁻ cells survived suggests that delivery of both grA and grB is compromised. This is consistent with the demonstration of the key role of the MPR in targeting grA from the trans-Golgi to early endosomes (Griffiths and Isaaz, 1993) and suggests that the same receptor acts for both granzymes.

Important Implications of the GrB-CI-MPR Interaction

The identification of a receptor for grB is significant in itself, and the discovery carries with it exciting possibilities for therapeutic manipulation. For example, in auto-immune disorders and in cases of transplant rejection, it would be beneficial to reduce levels of receptor expression. Further, we provide evidence that in cancer cells, the receptor expression is reduced, and predict that increased levels would promote sensitivity to killing by CTL/NK cells. However, the revelation that it is the previously described receptor for M6P-containing glycoproteins and IGF-II has a number of profound biological implications. For instance, it is interesting that the route of entry into the cell that is used by some toxins and viruses is the very pathway that will cause the demise of the infected cell (Brunetti et al., 1994; Zhu et al., 1995; Sandvig and Van Deurs, 1996; Molinari et al., 1997). Thus, it appears reasonable to assume that after entry of the virus in the cell, the receptor is somehow modified to affect or inhibit internalization of grB.he MPR has also been implicated in responses to some growth factors. Of particular note is the internalization and activation via the MPR of TGF-β, a cytokine that has long been known to act as an immunosuppressive agent (Kovacina et al., 1989). Perhaps TGF-β also acts in part by blocking the interaction of the receptor with grB and thus inhibiting entry. Similarly, local expression of other factors such as leukemia inhibitory factor may impact on responsiveness to grB. The present invention therefore enables a dissection of the structure function of CI-MPR in order to assess how the binding and internalization of its different ligands can regulate the pathways they affect and control upon their internalization

One of the most exciting aspects of the present work relates to the numerous reports that the CI-MPR receptor is a tumor suppressor. Loss of heterozygosity has been reported in a variety of human malignancies including hepatocarcinoma (De Souza et al., 1995) and aggressive early breast cancer (Chappell et al., 1997). Furthermore, the CI-MPR locus at 6q has been reported to be a hot spot for mutation in tumors including malignant melanoma (Millikin et al., 1991), ovarian cancer (Foulkes et al., 1993), non-Hodgkin lymphoma (Gaidano et al., 1992) and renal cell carcinoma (Morita et al., 1991). This has usually been interpreted on the basis of the CI-MPR regulating responses of cells to IGF-II signaling through the IGF-IR. However, our data suggest that tumors carrying mutated non-functional CI-MPR would also have an inherent resistance to the immune system, and this could play a critical role in the early escape of tumors and/or metastatic variants from host defenses. The results presented in Example 2 and the immune evasion of CI-MPR cells in vivo strongly support this contention. In addition, in Wilm's tumor and rhabdomyosarcomas, IGF-II is over-expressed and is believed to interfere with signaling through IGF-IR and the routing of lysosomal proteins (De Leon et al., 1996). It is also possible that the over-expressed factor could inhibit grB binding and internalization. An additional intriguing possibility is the recent observations that sCI-MPR is secreted by breast cancer lines and also primary metastic breast cancer cells (Confort et al., 1995). The secreted material could act as a sink for grB and thus prevent it from acting on the cell surface receptor. This would create a local immunosuppressive environment in a fashion analogous to secreted tumor necrosis factor receptor or Fas ligand. The results shown herein that apoptosis can be inhibited when cells are incubated wth CI-MPR once again support a critical role for MPR-dependent internalization of grB in cancer in animals and more generally in cellular homeostasy.

While the intricate regulation which is involved in the complex gateway which operates at CI-MPR, thereby affecting cellular homeostatis and possibly leading to disorders or diseases still needs to be formally determined, the present invention shows the critical importance thereof for a number of cellular processes. In addition, the present invention provides the intellectual framework for the design of experiments that will enable investigators to formally demonstrate them.

Inhibition of GrB Binding by M6P

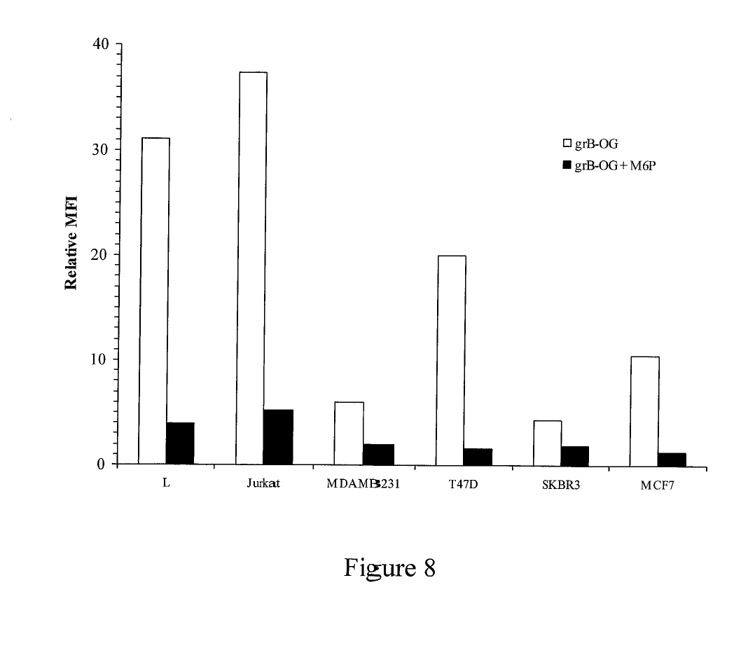

The binding and uptake of grB by the MPR is blocked by mannose-6-phosphate (FIG. 8). Cells were incubated at 37° C. with or without 20 mM M6P 15 minutes prior to addition of grB-OG. At a concentration of 20 mM, M6P significantly reduced binding of grB to the MPRs in all cell lines. These results verify that grB interacts with the MPR via its M6P ligand binding sites.

GrB/AD-Mediated Apoptosis

The next stage was to determine if any grB-MPR interaction on the cell surface resulted in grB biological activity within the cell; proteolytic activation of caspases giving rise to apoptotic events.

In normal cells, the distribution of phospholipids is asymetric, with the inner membrane containing anionic phospholipids (such as phosphatidylserine) and the outer membrane having mostly neutral phospholipids. In apoptotic cells, the amount of phosphatidylserine on the outer surface of the membrane increases. Annexin V, a calcium-dependent phospholipid-binding protein with a high affinity for phosphatidylserine, will therefore bind to the surface of apoptotic cells (Guide to Cell Proliferation and Apoptosis Methods, 2000, Roche Diagnostics Corporation). Labeled-annexin V binding can then also be detected by flow cytometry.

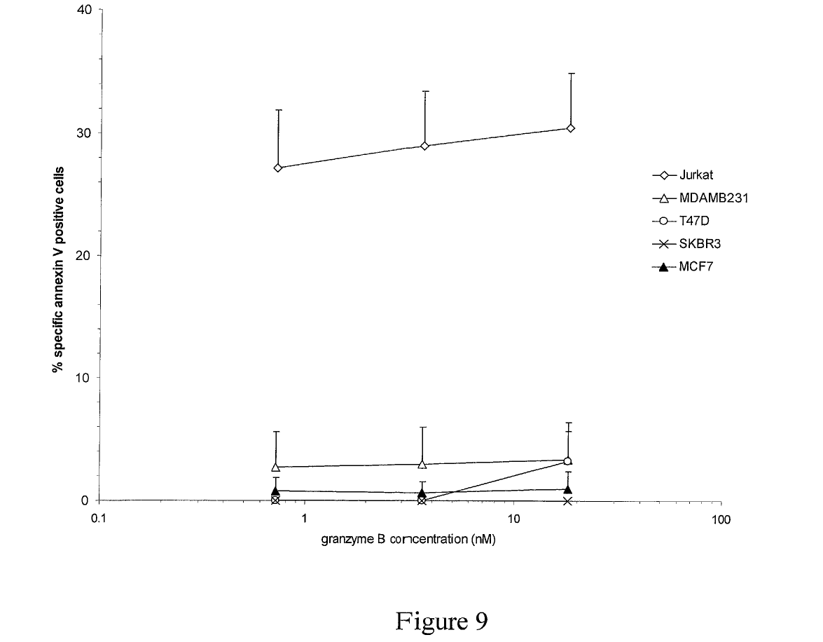

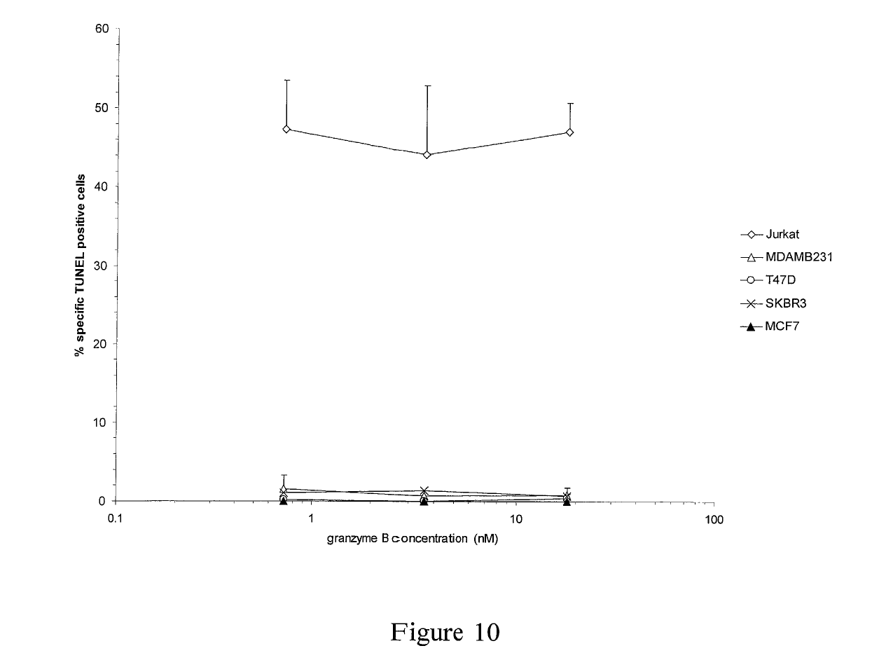

Cells were treated for 3 hr with varying concentrations of grB and replication-deficient adenovirus (AD) and afterwards assessed for grB-induced DNA fragmentation and phosphatidylserine externalization. Adenovirus is required to mediate the release of grB into the cytoplasm after being taken up by receptor-mediated endocytosis (Motyka et al., 2000). Jurkat cells were used as a positive control for both assays. As expected, only the Jurkat cells showed significant TUNEL and annexin V labeling (FIGS. 9 and 10). Interestingly, all the carcinoma cell lines were resistant to grB and AD-mediated DNA fragmentation and did not bind annexin V (FIGS. 9 and 10).

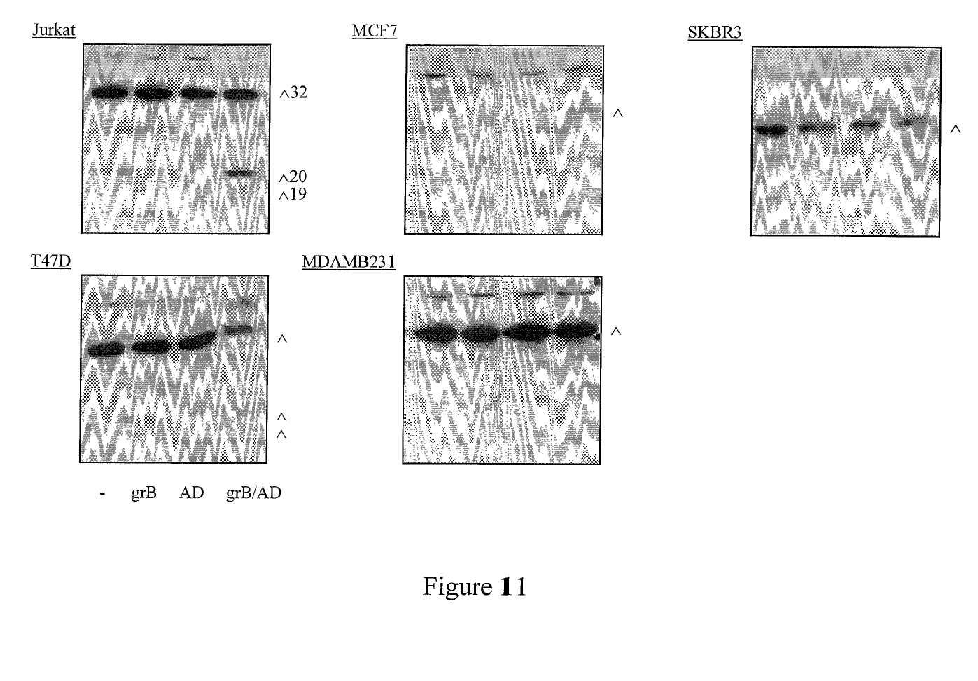

We also monitored the cleavage of caspase-3 by Western blot analysis, since the activation of caspase-3 has been connected with the onset of apoptotic events such as DNA fragmentation and phosphatidylserine externalization through the cleavage of various cellular proteins such as the inhibitor (ICAD) of caspase-activated DNase (Heibein et al., 1999). Most important, monitoring of grB-mediated caspase-3 cleavage allowed us to establish whether grB was actually getting into the cells since caspase-3 is a direct substrate for grB in vitro (Barry et al., 2000). The positive control was once again Jurkat cells and we also possessed a negative control, the caspase-3 deficient MCF-7 (Barry et al., 2000). We were able to observe cleavage of procaspase-3 (32-kDa) only in Jurkat (positive control) and T47-D cells to 20- and 19-kDa fragments (FIG. 11), indicating that grB is getting into the T47-D cells. Procaspase-3 was identified in MDA-MB-231 and SK-BR-3 cells but we were unable to observe any cleavage, strongly suggesting that grB is not getting into the cells. This relates nicely to the reduced level of expression of CI-MPR in the two cell lines. If there is no receptor for grB to bind to, it cannot enter. MCF-7 cells are resistant to grB/AD-mediated DNA fragmentation and membrane changes because they do not have caspase-3 and therefore cannot initiate caspase-dependent apoptotic pathways.

### EXAMPLE2

### Involvement of the grB Internalization Mechanism and the Activity of grB on Immune Evasion by Cells

Cell Lines and Reagents

Jurkat cells were grown in RPMI 1640 medium (Gibco BRL Life Technologies Inc.) supplemented with 10% fetal calf serum (FCS) (Hyclone), 25 mM HEPES, 100 μM 2-mercaptoethanol, 100 μg of pencillin per ml, and 100 μg of streptomycin per ml (RHFM). MCF-7 cells were cultured in RHFM supplemented with 100 μM nonessential amino acids (Gibco BRL Life Technologies Inc.). Murine L cells were grown in Dulbecco's modified Eagle's medium (DMEM, Gibco) supplemented with 10% FCS and with 2 mM L-glutamine and 100 μg/ml penicillin/streptomycin. MS and MS9II cells were maintained as previously described (Watanabe et al., 1990). T47D and MDA-MB-231 cells were cultured in Iscove's Modified Dulbecco's Medium (IMDM, Gibco) supplemented with 10% FCS glutamine and 100 μg/ml penicillin/streptomycin. SK-BR-3 cells were in DMEM supplemented with 10% FCS and with 2 mM L-glutamine and 100 μg/ml penicillin/streptomycin. All cell lines were passaged by trypsinization, but prior to any analysis they removed from culture flasks with 2% EDTA/PBS solution. Rabbit antisera with specificity for the human CI-MPR (Wood et al., 1991) or CD-MPR (Ma et al., 1991) have been described. Goat anti-rabbit-FITC was obtained from Jackson ImmunoResearch and streptavidin-cychrome from PharMingen. Staurosporine and M6P were purchased from Sigma.

Flow Cytometric Analysis of Cell Surface MPR Expression

Cells (1×10⁵) were incubated for 60 min at 4° C. with a 200-fold dilution of rabbit anti-Cl- or CD-MPR and washed twice. Cells were then incubated with a 100-fold dilution of goat-anti-rabbit-FITC and washed twice. 0.1% BSA/PBS buffer was used throughout the procedure. Flow cytometric analysis was performed on a Becton Dickinson FACScanθ flow cytometer equipped with an argon-ion laser with 15 mW of excitation at 488 nm. Emission wavelengths were detected through the FL1. Data were acquired on 10,000 cells per sample with light scatter signals at linear gain and fluorescence signals at logarithmic gain.

Internal Labeling of CI-MPR

Cells (2×10⁵) were fixed in 4% paraformaldehyde/PBS for 10 min., and permeabilized in 0.1% saponin/PBS for 15 min and washed. Cells were then blocked in 4% normal donkey serum. Cells were then incubated for 60 min at 37EC with a -fold dilution of rabbit anti-CI-MPR and washed twice. Cells were blocked again with 4% normal donkey serum. Cells were then incubated with a 100-fold dilution of goat-anti-rabbit FITC for 30 min and washed twice. Flow cytometric analysis was performed on a Becton Dickinson FACScanθ flow cytometer equipped with an argon-ion laser with 15 mW of excitation at 488 nm. Emission wavelengths were detected through the FL1. Data were acquired on 10,000 cells per sample with light scatter signals at linear gain and fluorescence signals at logarithmic gain.

GrB Binding and Uptake Assays

Cells (1×10⁵) were washed twice and resuspended in buffer before addition of grB-biotin or grB-OG in a final volume of 20 μl for a 30-60 min incubation and washed again twice. In the case of grB-biotin, cells were then incubated with streptavidin-cychrome for 20 min and washed twice. In order to measure cell surface binding, all steps of the binding assay were carried out at 4° C. to prevent intracellular uptake of grB. 0.1% BSA/PBS buffer was used throughout the procedure. All steps of the grB uptake assay were carried out at 37° C. Flow cytometric analysis was performed on a Becton Dickinson FACScanθ flow cytometer equipped with an argon-ion laser with 15 mW of excitation at 488 nm. Emission wavelengths were detected through the FL1 and FL3 channel for grB-OG and grB-biotin, respectively. Data were acquired on 10,000 cells per sample with light scatter signals at linear gain and fluorescence signals at logarithmic gain. Results are presented as the relative MFI, which represents the MFI of the grB-labeled population minus the MFI of the unlabeled population.

M6P Inhibition Studies

M6P at 20 mM was added to cells for 15 min at 37° C. before the addition of labeled grB.

Apoptosis Induction

Cells were resuspended at 10⁶ cells/ml in 0.1% BSA/DMEM. Adenovirus (10 PFU/cell) and granzyme B (grB) were added directly to the cell suspension. Cells were incubated at 37° C. for 3 h. For staurosporine killing, cells were resuspended at 10⁶ cells/ml in their normal growth medium and staurosporine (2.5 μM) was added directly to the cell suspension. Cells were incubated at 37° C. for 2, 4 and 8 h.

Flow Cytometric Analysis of DNA Fragmentation

DNA fragmentation was monitored by flow cytometric analysis via the terminal deoxynucleotidyltransferase-mediated dUTP-biotin nick end labeling (TUNEL) method. Flow cytometric analysis was performed on a Becton Dickinson FACScanθ flow cytometer equipped with an argon-ion laser with 15 mW of excitation at 488 nm. Emission wavelengths were detected through the FL1 channel. Data were acquired on 10,000 cells per sample with light scatter signals at linear gain and fluorescence signals at logarithmic gain. Percent specific TUNEL positive cells was calculated as follows: [(% positive labeling cells with grB and AD−% positive labeling cells without grB and AD)/(AD−% positive labeling cells without grB and AD)]×100.

Detection of Phosphatidylserine Externalization by Flow Cytometry

Phosphatidylserine externalization was monitored using the ApoAlertθ annexin V apoptosis kit (Clontech) according to the protocol provided by the manufacturer. Flow cytometric analysis was performed on a Becton Dickinson FACScanθ flow cytometer equipped with an argon-ion laser with 15 mW of excitation at 488 nm. Emission wavelengths were detected through the FL1 channel. Data were acquired on 10,000 cells per sample with light scatter signals at linear gain and fluorescence signals at logarithmic gain. Percent specific Annexin V positive cells was calculated as follows: [(% positive labeling cells with grB and AD−% positive labeling cells without grB and AD)/(AD−% positive labeling cells without grB and AD)]×100.

Immunoblotting

Cellular lysates were collected by directly harvesting 10⁵ cells into 100 μl of sodium dodecyl sulfate-polyacrylamide gel electrophoresis (SDS-PAGE) gel loading buffer and subjected to SDS-PAGE analysis. Proteins were transferred to nitrocellulose (Micron Separations Inc.) by using a semidry transfer apparatus (Tyler Corp.) for 1 h at 150 mA. Membranes were blocked in phosphate-buffered saline (PBS) containing 0.1% Tween 20 (ICN Biomedicals Inc.) and 5% skim milk for 16 h. Caspase 3 was detected using polyclonal rabbit anti-caspase 3 at a dilution of 1:10,000. The membrane was incubated with the primary antibody for 2 h, after which the blot was washed three times in PBS containing 0.1% Tween 20. The membranes were probed with a horseradish peroxidase-conjugated goat anti-rabbit secondary antibody (Bio-Rad Laboratories) at a 1:20,000 dilution. Transferred proteins were visualized with a chemiluminescence detection system (Amersham Inc.).

Results

Binding of GrB Correlates to Cell Surface MPR Expression

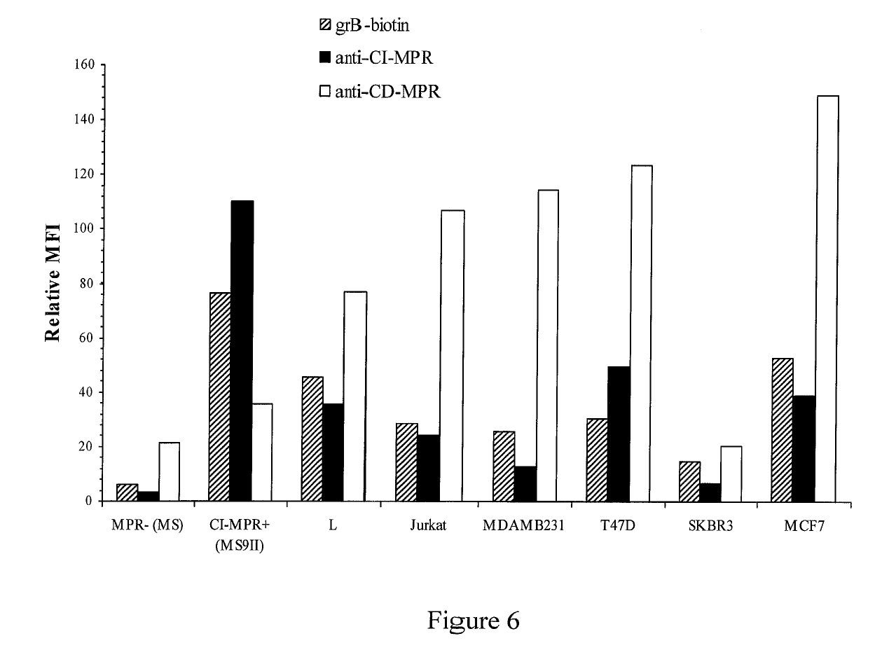

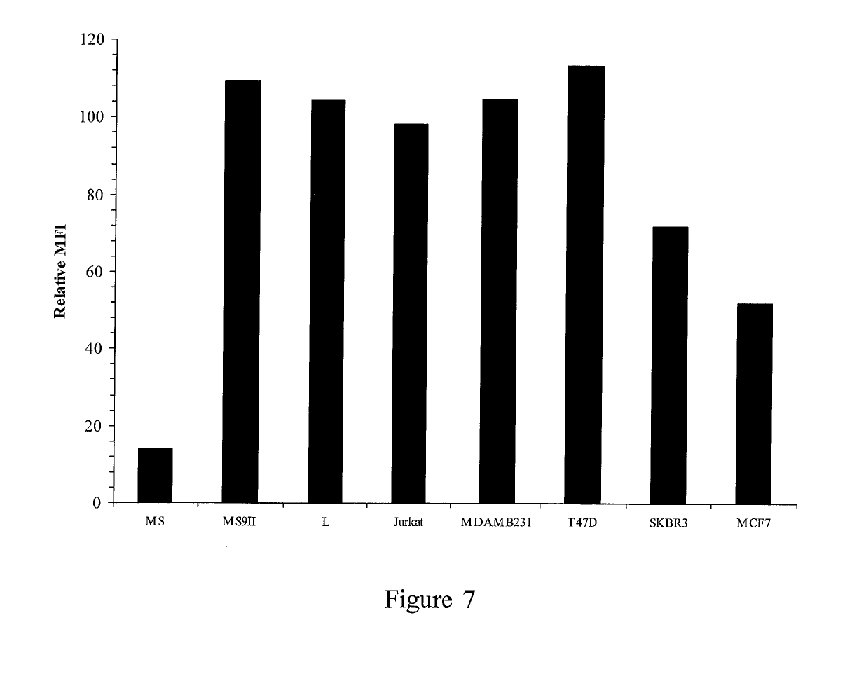

As human cells (and other animal cells as well) possess two types of mannose-6-phosphate receptors, the ˜270 kDa CI-MPR and the 46 kDa CD-MPR, that both have a high binding affinity for proteins with phosphomannosyl residues (Motyka et al., 2000). The binding of grB-biotin was compared with the level of expression of cell surface MPR by flow cytometric analysis using antisera specific for the CI- and CD-MPR. All the breast carcinoma cell lines, as well as Jurkat cells, wild-type L cells and L cells that were either deficient in the expression of CI-MPR (MS) or that were transfected to overexpress the CI-MPR (MS9II) were treated as described above in Example 2. We found a direct correlation between cell surface MPR expression and grB binding (FIG. 6). In addition, we identified two carcinoma cell lines, SK-BR-3 and MDA-MB-231 that have very low levels of expression of cell surface CI-MPR, even comparable to MS (CI-MPR-) cells. For SK-BR-3 cells, this is in agreement with the results obtained from internal anti-CI-MPR labeling, which also displayed reduced levels of CI-MPR (FIG. 7). We also observed decreased levels of total CI-MPR in MCF-7 cells. In contrast, MDA-MB-231 cells displayed steady-state levels of CI-MPR expression, suggesting that MDA-MB-231 cells can avoid grB/CI-MPR-mediated apoptosis by decreasing expression of cell surface CI-MPR.

Furthermore, we observed high levels of expression of cell surface CD-MPR in three of the breast cancer cell lines, T47-D, SK-BR-3 and MDA-MB-231 (FIG. 6). The level of expression of cell surface CD-MPR in the MCF-7 cell line was similar to L cells that were transfected to overexpress the CD-MPR (data not shown). A likely explanation for the significant binding of grB despite reduced surface CI-MPR expression in the MDA-MB-231 cell line is that grB is capable of binding also to the CD-MPR. A striking difference between CD-MPR and CI-MPR is the inability of the former to mediate internalization of M6P containing ligands (Sandholzer et al., 2000). Only under specific conditions such as high ligand concentration and strong overexpression of the receptor, is a low endocytic activity of CD-MPR detectable (Watanabe et al., 1990). We cannot rule out a minor CD-MPR pathway; perhaps a local concentration of grB can be achieved at the CTL-target interface such that some uptake can occur via the CD-MPR (Motyka et al., 2000). The inability of overexpressed CD-MPR to endocytose grB could be beneficial for a tumor cell because the high levels of surface CD-MPR could act as a sink for grB and thus prevent it from acting on the CI-MPR. Thus, an increased expression of CD-MPR may be yet another method to lower or inhibit grB-mediated apoptosis.

Discussion

Taken together, the findings reported in Example 2 demonstrate that all the breast carcinoma cell lines use several strategies to evade granule purified grB-mediated apoptosis.

We observed a clearly reduced level of expression of cell surface CI-MPR in SK-BR-3 and MDA-MB-231 cells (FIG. 6). If the binding and internalization of grB by receptor-mediated endocytosis is necessary for apoptosis, then grB that does not bind to the cell surface should be ineffective at killing. Consistent with this assumption, we did not observe any caspase-3 activation in the presence of grB and AD. Since grB has been shown in vitro to cleave caspase-3 (Barry et al., 2000), this strongly implies that grB is not being internalized. However, in the MDA-MB-231 cell line we observed a high level of grB binding. A striking difference between the MDA-MB-231 and SK-BR-3 cell line is the high level of expression of cell surface CD-MPR in the latter. The most obvious explanation is that CD-MPR also binds grB resulting in increased grB binding, but in turn preventing its' entry via the large receptor. Furthermore it appears that CI-MPR is actively prevented from being externalized on the surface of MDA-MB-231 cells, as they appear to have relatively normal levels of total CI-MPR expression (FIG. 7). Undoubtedly the reduction in cell surface CI-MPR expression plays a significant role in preventing the entry of grB.

The levels of expression of cell surface CI-MPR were relatively normal in the MCF-7 and T47-D cell lines and both in turn bound grB at significant levels (FIG. 6). Interestingly, both cell lines are estrogen receptor-positive cell lines and it has been shown that estradiol specifically decreases the steady-state level of the CI-MPR protein and mRNA (Mathieu et al., 1991). Therefore in vivo, hormone-dependent breast cancer cells could down-regulate the CI-MPR, in turn preventing grB-mediated cell killing. It would be interesting to culture these cell lines in the presence of estrogens anti-estrogens such as tamoxifen and then examine MPR expression and grB binding. The estradiol-induced down-regulation of the CI-MPR was not observed in the ER-negative MDA-MB-231 cell line.

Yet, as we did for MDA-MB-231 cells, we observed a high level of expression of surface CD-MPR for T47-D and MCF-7 cells. Based on these results, we propose that CD-MPR is capable of binding grB but unable to endocytose it, thus elevated levels of the small receptor could act as a sink for grB, preventing it from interacting with the CI-MPR. This would create a local immunosuppressive environment in a fashion analogous to secreted tumor necrosis factor receptor or Fas ligand (Motyka et al., 2000). The incapacity of CD-MPR to endocytose grB is supported by the results presented in Example 1 using cells which over-express CD-MPR (e.g. which are essentially unable to internalize grB and unable to undergo grB-mediated apoptosis).

We also confirmed that MCF-7 and T47-D cells possess mutations in the apoptotic pathway that also protect against grB-mediated cell death. MCF-7 cells express no caspase 3 due to a deletion within exon three that is critical for correct processing of the mRNA. Caspase-3 is required for DNA fragmentation and morphological changes associated with apoptosis (Janicke et al. 1998, J. Biol. Chem. 273:9357-9360). When MCF-7 cells were treated with granzyme B and adenovirus, no DNA-fragmentation or phosphatidylserine externalization could be detected (FIGS. 9 and 10). However, previous studies from our laboratory have demonstrated that MCF-7 cells are not resistant to CTL-mediated killing. We have demonstrated that mitochondrial collaspe and cytochrome c release, also hallmarks of apoptosis, occur independent of caspase activation during granule-mediated apoptosis (Barry et al., 2000). These results reiterate that caspase 3 is necessary for grB-mediated DNA fragmentation and phosphatidylserine externalization.

Internalized grB was responsible for the cleavage of caspase-3 (FIG. 11) and Bid (data not shown) in T47-D cells. Direct cleavage of Bid bypasses the need for caspase-8 activation of Bid, a major effector molecule in Fas-mediated apoptosis (Barry et al., 2000). Yet we were unable to detect any phosphatidylserine exposure or DNA fragmentation in T47-D cells following treatment with granzyme B and adenovirus (FIGS. 9 and 10), indicating that the T47-D cell line is somehow altered downstream of caspase-3 activation in the grB-mediated apoptotic pathway.

Another possible evasive strategy, which was not examined in this study, is the secretion by tumor cells of ligands capable of either saturating the cell surface CI-MPR or sequestering grB. In either case, grB is prevented from acting on the CI-MPR. It has been shown that insulin-like growth factors (IGFS) stimulate the release of sCI-MPR from MCF-7 cells (Confort et al., 1995). Both hormone-dependent and independent breast cancer cells secrete more procathepsin-D, a CI-MPR ligand, than normal mammary cells cultured in the same conditions (Mathieu et al., 1991).

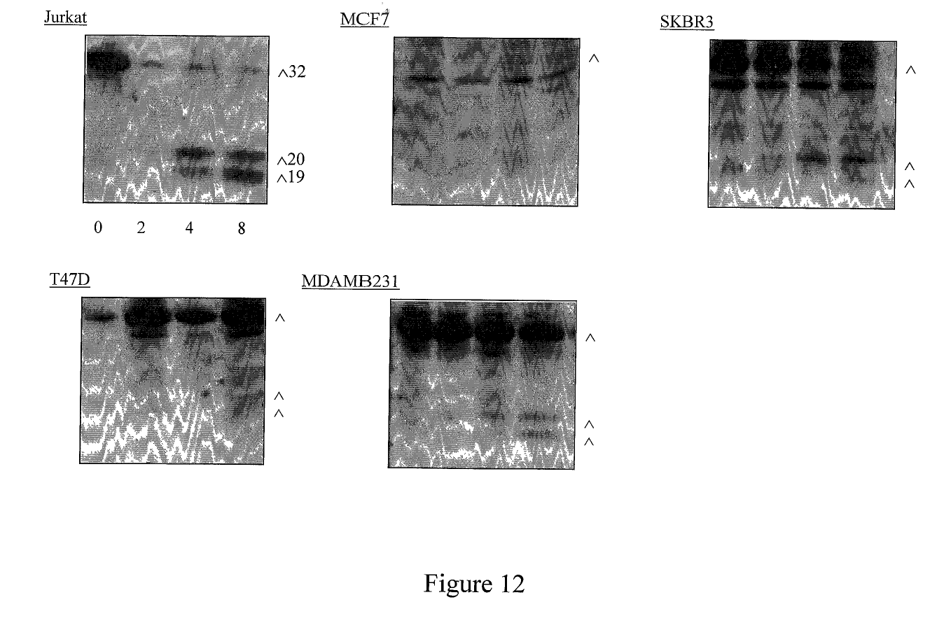

Because of the negative results observed with grB-mediated DNA fragmentation and membrane changes, it was essential to determine if the various breast cancer cell lines could die by apoptosis (i.e. could the grB-dependent apoptosis defect in fact be dependent on a defect in a downstream factor involved in apoptosis). For example, although caspase-3 deficient MCF-7 cells are resistant to grB-mediated DNA fragmentation and phosphatidylserine externalization, we know that CTLs can destroy MCF-7 cells via a caspase-3 independent pathway (Barry et al., 2000). Apoptosis can be induced by various stimuli including DNA-damaging anticancer drugs and the protein kinase inhibitor staurosporine. While experiments involving staurosporine are ongoing, preliminary results indicate that procaspase-3 is cleaved in all the cells after treatment with staurosporine (FIG. 12). Thus, caspase 3 is activable in these cells. It follows that if grB was internalized in these cells, the CTL-dependent killing induced by grB could be activated. It follows that restoration of grB internalization in such cells could enable CTL-killing thereof. Thus, increasing internalization of grB in cancer cells initially having a decreased level thereof, together with a vaccine therapy could be used in a CTL-mediated tumor killing therapy for such cancers.

We also cannot neglect the possibility that in any one of the breast carcinoma cell lines, the CI-MPR gene sequence may be mutated, e.g., amino-acid substitutions. Previous results indicate no mutation of the CI-MPR coding sequence in MCF-7 and MDA-MB-231 cells (Ray et al., 2000). Nonetheless, this does not rule out post-translational modifications that could also cause the receptor to become nonfunctional. The CI-MPR receptor sequences in SK-BR-3 and T47-D cell lines have yet to be characterized. As noted above, both MCG-7 and T47-D possess mutations in factors involved in the apoptotic pathway, thus reinforcing the possibility that mutation could be present in genes more directly involved in grB internalization and triggering of grB-mediated apoptosis. In any event, without wanting to be limited to a particular theory, the present invention clearly and unequivocally identifies essential factors in grB-mediated apoptosis and CTLs. It is clear that cells have divised multiple non-exclusive means to override the grB-mediated apoptotic pathway. It has herein been demonstrated that grB internalization through its receptor is a cornerstone even for proper cellular homeostasy. The instant invention has thus important implications not only in apoptosis and CTL-induced apoptosis, but in all diseases, conditions, or normal cellular processes which are dependent on the binding and/or internalization of grB by cells. Non-limiting examples thereof include cancer, auto-immune diseases, transplantation and viral infections and more broadly, disease or conditions associated with a deregulation of grB internalization in cells (e.g. multiple sclerosis).

### EXAMPLE 3

### Conservation of the Sequences of the Present Invention Throughout Evolution

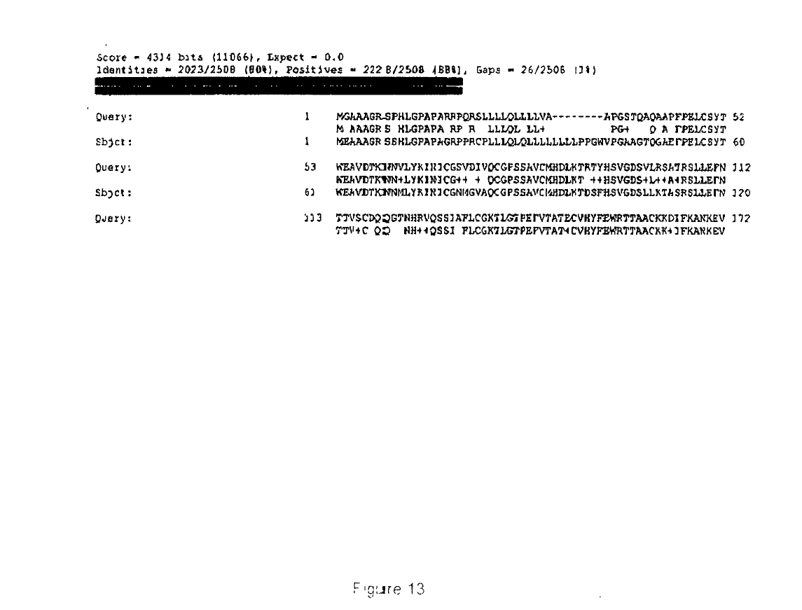

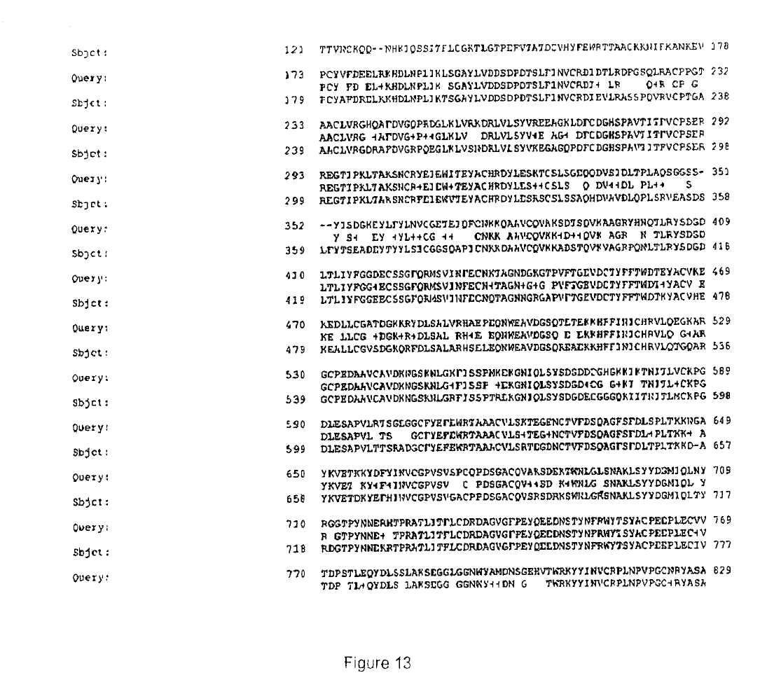

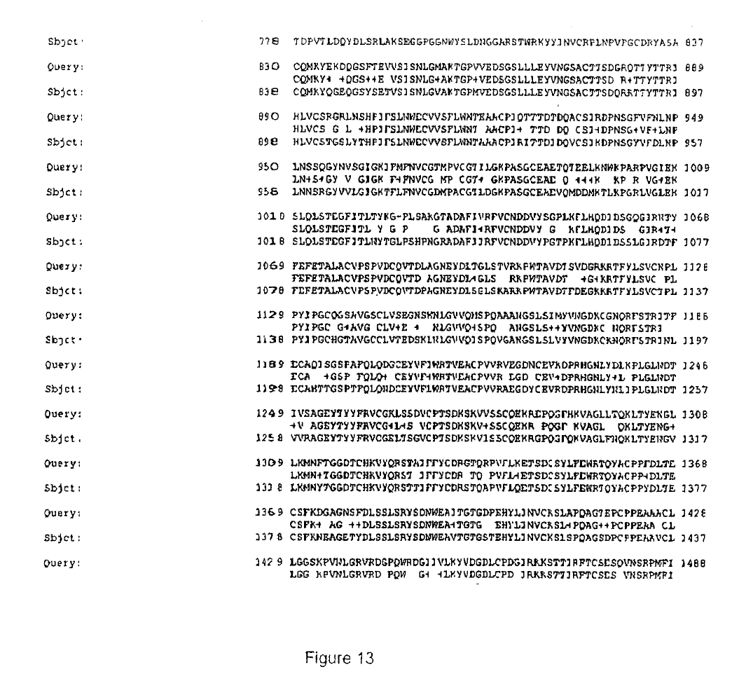

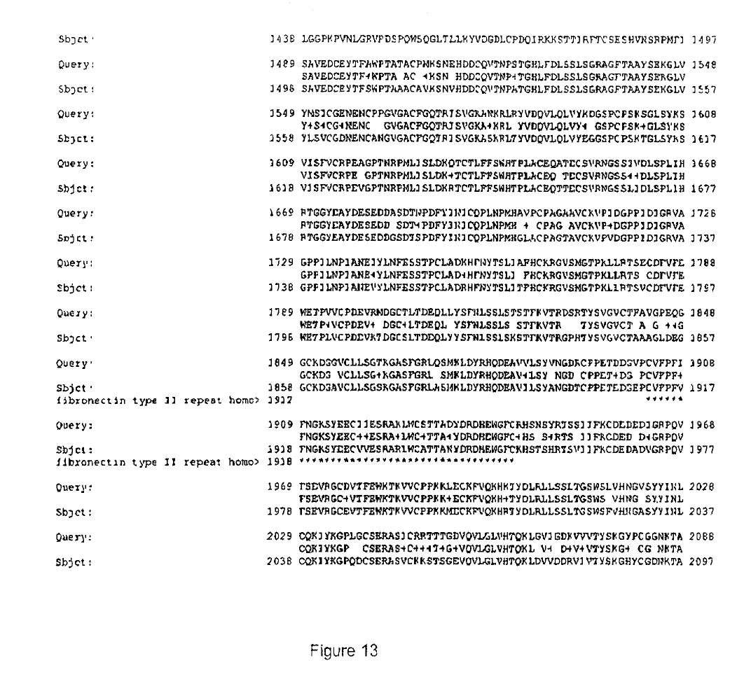

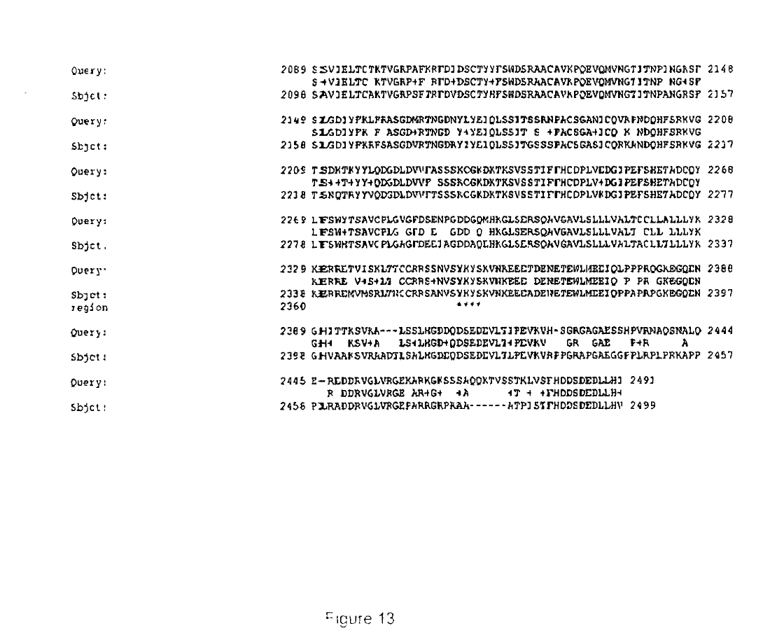

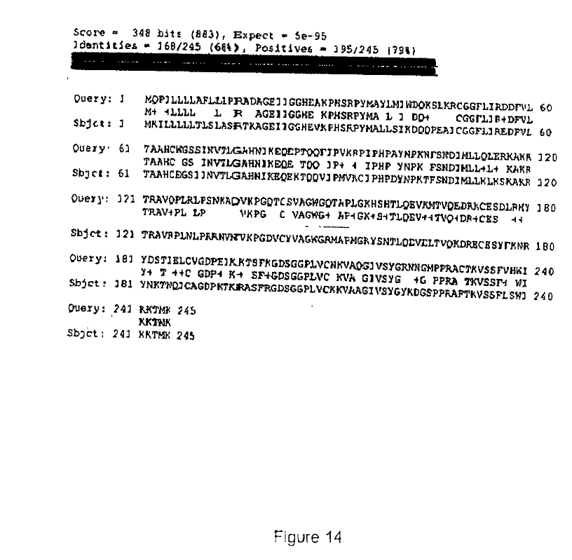

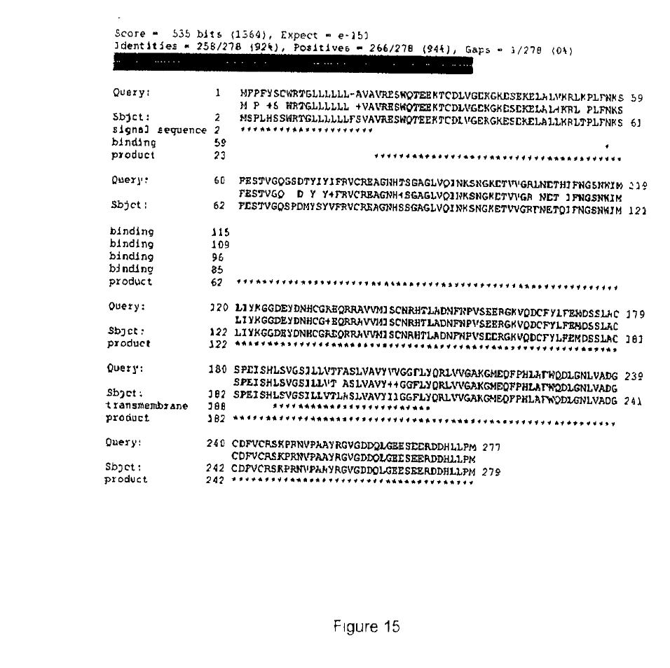

The methods and assays of the present invention can be performed with sequences from animals in general, preferably mammalian species, in view of the significant conservation of their amino acid and nucleotide sequences and of experiments demonstrating functional complementation between evolutionary divergent animal species. Three alignments are shown in FIGS. 13-15.

### CONCLUSION

We have demonstrated that the CI-MPR is a receptor for grB on the target cell surface and that this recognition is necessary for the efficient apoptosis of target cells mediated by granule-purified grB or by CTL. The modulation of the CI-MPR on the target cell surface or the modulation of the level and/or activity of internalized grB (or grA) would thus have profound repercussions on the ability of CTL to induce apoptosis by the grB-mediated pathway.

In conclusion, we have identified elements of a potential model to describe how breast carcinoma cell lines can evade grB-mediated apoptosis. Firstly, the down-regulation of surface CI-MPR and/or up-regulation of surface CD-MPR, thus preventing the entry of grB into the cell. Secondly, key mutations or deletions in apoptotic pathways that prevent grB-mediated apoptosis. The next step is to apply our model to primary tissue to determine if similar mechanisms are used in order to avoid CTL-mediated apoptosis in vivo.

Although the present invention has been described hereinabove by way of preferred embodiments thereof, it can be modified without departing from the spirit and nature of the subject invention as defined in the appended claims.

What is claimed is:

1. A method of inhibiting a disease or disorder in an individual associated with an enhanced internalization of granzyme B (grB), comprising administering to said individual an effective amount of an agent which inhibits grB binding to the cation-independent mannose 6-phospate receptor (CI-MPR) and/or internalization of grB thereby.

2. The method of claim 1, wherein the agent is selected from the group consisting of antisense polynucleotide to the CI-MPR, soluble cation-dependent mannose 6-phospate receptor (CD-MPR), soluble CI-MPR, a mannose 6-phosphate containing molecule, an agent which increases the level of CD-MPR on the outer membrane of a cell associated with said disease or disorder, and a ligand which saturates CI-MPR or sequesters grB.

3. The method of claim 2, wherein said ligand is an antibody which binds to CI-MPR.

4. A method of increasing granzyme B internalization in a cell comprising increasing the level and/or activity of CI-MPR on the outer membrane of said cell.

5. The method of claim 4, wherein said increased level of CI-MPR on the outer membrane of said cell is effected by increasing expression of a gene encoding CI-MPR.

6. The method of claim 4, wherein said increased level of CI-MPR on the outer membrane is effected by administration to the cell of a polynucleotide encoding the CI-MPR receptor.

7. The method of claim 6, wherein the polynucleotide is delivered to said cell.

8. The method of claim 4, wherein said increased increased level of CI-MPR on the outer membrane is effected by of CI-MPR on the outer membrane is effected by increasing externalization of intracellularly localized CI-MPR.

9. A method for identifying an agent that modulates CI-MPR-dependent internalization of granzyme B in a cell comprising contacting a CI-MPR, or fragment thereof in the presence or absence of a candidate compound and assaying a biological function of said CI-MPR, or fragment thereof, wherein a modulator of said CI-MPR-dependent internalization of granzyme B is selected when said biological function is measurably different in the presence of said candidate agent as compared to in the absence thereof.

10. A method for identifying an agent that modulates CI-MPR-dependent internalization of granzyme B in a cell comprising contacting a CI-MPR, or fragment thereof with a mannose 6-phosphate-containing molecule in the presence or absence of a candidate compound and assaying a biological function of said CI-MPR, wherein a modulator of said CI-MPR-dependent internalization of granzyme B is selected when said biological function is measurably different in the presence of said candidate agent as compared to in the absence thereof.

11. The method of claim 10, wherein said mannose 6-phosphate containing molecule is granzyme B (grB), and wherein said biological function is a biological function effected by grB.


SEQUENCE LISTING (6 sequences)

SEQ ID NO: 1 - DNA, 9090 bp, homo sapiens CIMPRna
  variation (1197): n = a or g
cgagcccagtcgagccgcgctcacctcgggctcccgctccgtctccacctccgcctttgccctggcggcgcgaccccgtcccggcgcggcccccagcagtcgcgcgccgttagcctcgcgcccgccgcgcagtccgggcccggcgcgatgggggccgccgccggccggagcccccacctggggcccgcgcccgcccgccgcccgcagcgctctctgctcctgctgcagctgctgctgctcgtcgctgccccggggtccacgcaggcccaggccgccccgttccccgagctgtgcagttatacatgggaagctgttgataccaaaaataatgtactttataaaatcaacatctgtggaagtgtggatattgtccagtgcgggccatcaagtgctgtttgtatgcacgacttgaagacacgcacttatcattcagtgggtgactctgttttgagaagtgcaaccagatctctcctggaattcaacacaacagtgagctgtgaccagcaaggcacaaatcacagagtccagagcagcattgccttcctgtgtgggaaaaccctgggaactcctgaatttgtaactgcaacagaatgtgtgcactactttgagtggaggaccactgcagcctgcaagaaagacatatttaaagcaaataaggaggtgccatgctatgtgtttgatgaagagttgaggaagcatgatctcaatcctctgatcaagcttagtggtgcctacttggtggatgactccgatccggacacttctctattcatcaatgtttgtagagacatagacacactacgagacccaggttcacagctgcgggcctgtccccccggcactgccgcctgcctggtaagaggacaccaggcgtttgatgttggccagccccgggacggactgaagctggtgcgcaaggacaggcttgtcctgagttacgtgagggaagaggcaggaaagctagacttttgtgatggtcacagccctgcggtgactattacatttgtttgcccgtcggagcggagagagggcaccattcccaaactcacagctaaatccaactgccgctatgaaattgagtggattactgagtatgcctgccacagagattacctggaaagtaaaacttgttctctgagcggcgagcagcaggatgtctccatagacctcacaccacttgcccagagcggaggttcntcctatatttcagatggaaaagaatatttgttttatttgaatgtctgtggagaaactgaaatacagttctgtaataaaaaacaagctgcagtttgccaagtgaaaaagagcgatacctctcaagtcaaagcagcaggaagataccacaatcagaccctccgatattcggatggagacctcaccttgatatattttggaggtgatgaatgcagctcagggtttcagcggatgagcgtcataaactttgagtgcaataaaaccgcaggtaacgatgggaaaggaactcctgtattcacaggggaggttgactgcacctacttcttcacatgggacacggaatacgcctgtgttaaggagaaggaagacctcctctgcggtgccaccgacgggaagaagcgctatgacctgtccgcgctggtccgccatgcagaaccagagcagaattgggaagctgtggatggcagtcagacggaaacagagaagaagcattttttcattaatatttgtcacagagtgctgcaggaaggcaaggcacgaggntgtcccgaggacgcggcagtgtgtgcagtggataaaaatggaagtaaaaatctgggaaaatttatttcctctcccatgaaagagaaaggaaacattcaactctcttattcagatggtgatgattgtggtcatggcaagaaaattaaaactaatatcacacttgtatgcaagccaggtgatctggaaagtgcaccagtgttgagaacttctggggaaggcggttgcttttatgagtttgagtggcncacagctgcggcctgtgtgctgtctaagacagaaggggagaactgcacggtctttgactcccaggcagggttttcttttgacttatcacctctcacaaagaaaaatggtgcctataaagttgagacaaagaagtatgacttttatataaatgtgtgtggcccggtgtctgtgagcccctgtcagccagactcaggagcctgccaggtggcaaaaagtgatgagaagacttggaacttgggtctgagtaatgcgaagctttcatattatgatgggatgatccaactgaactacagaggcggcacnccctataacaatgaaagacacacaccgagagctacgctcatcacctttctctgtgatcgagacgcgggagtgggcttccctgaatatcaggaagaggataactccacctacaacttccggtggtacaccagctatgcctgcccggaggagcccctggaatgcgtagtgaccgacccctccacgctggagcagtacgacctctccagtctggcaaaatctgaaggtggccttggaggaaactggtatgccatggacaactcaggggaacatgtcacgtggaggaaatactacattaacgtgtgtcggcctctgaatccagtgccgggctgcaaccgatatgcatcggcttgccagatgaagtatgaaaaagatcagggctccttcactgaagtggtttccatcagtaacttgggaatggcaaagaccggcccggtggttgaggacagcggcagcctccttctggaatacgtgaatgggtcggcctgcaccaccagcgatggcagacagaccacatataccacgaggatccatctcgtctgctccaggggcaggctgaacagccaccccatcttttctctcaactgggagtgtgtggtcagtttcctgtggaacacagaggctgcctgtcccattcagacaacgacggatacagaccaggcttgctctataagggatcccaacagtggatttgtgtttaatcttaatccgctaaacagttcgcaaggatataacgtctctggcattgggaagatttttatgtttaatgtctgcggcacaatgcctgtctgtgggaccatcctgggaaaacctgcttctggctgtgaggcagaaacccaaactgaagagctcaagaattggaagccagcaaggccagtcggaattgagaaaagcctccagctgtccacagagggcttcatcactctgacctacaaagggcctctctctgccaaaggtaccgctgatgcttttatcgtccgctttgtttgcaatgatgatgtttactcagggcccctcaaattcctgcatcaagatatcgactctgggcaagggatccgaaacacttactttgagtttgaaaccgcgttggcctgtgttccttctccagtggactgccaagtcaccgacctggctggaaatgagtacgacctgactggcctaagcacagtcaggaaaccttggacggctgttgacacctctgtcgatgggagaaagaggactttctatttgagcgtttgcaatcctctcccttacattcctggatgccagggcagcgcagtggggtcttgcttagtgtcagaaggcaatagctggaatctgggtgtggtgcagatgagtccccaagccgcggcgaatggatctttgagcatcatgtatgtcaacggtgacaagtgtgggaaccagcgcttctccaccaggatcacgtttgagtgtgctcagatatcgggctcaccagcatttcagcttcaggatggttgtgagtacgtgtttatctggagaactgtggaagcctgtcccgttgtcagagtggaaggggacaactgtgaggtgaaagacccaaggcatggcaacttgtatgacctgaagcccctgggcctcaacgacaccatcgtgagcgctggcgnatacacttattacttccgggtctgtgggaagctttcctcagacgtctgccccacaagtgacaagtccaaggtggtctcctcatgtcaggaaaagcgggaaccgcagggatttcacaaagtggcaggtctcctgactcagaagctaacttatgaaaatggcttgttaaaaatgaacttcacggggggggacacttgccataaggtttatcagcgctccacagccatcttcttctactgtgaccgcggcacccagcggccagtatttctaaaggagacttcagattgttcctacttgtttgagtggcgaacgcagtatgcctgcccacctttcgatctgactgaatgttcattcaaagatggggctggcaactccttcgacctctcgtccctgtcaaggtacagtgacaactgggaagccatcnctgggacgggggacccggagcactacctcatcaatgtctgcaagtctctggccccgcaggctggcactgagccgtgccctccagaagcagccgcgtgtctgctgggtggctccaagcccgtgaacctcggcagggtaagggacggacctcagtggagagatggcataattgtcctgaaatacgttgatggcgacttatgtccagatgggattcggaaaaagtcaaccaccatccgattcacctgcagcgagagccaagtgaactccaggcccatgttcatcagcgccgtggaggactgtgagtacacctttgcctggcccacagccacagcctgtcccatgaagagcaacgagcatgatgactgccaggtcaccaacccaagcacaggacacctgtttgatctgagctccttaagtggcagggcnggattcacagctgcttacagcgagaaggggttggtttacatgagcatctgtggggagaatgaaaactgccctcctggcgtgggggcctgctttggacagaccaggattagcgtgggcaaggccaacaagaggctgagatacgtggaccaggtcctgcagctggtgtacaaggatgggtccccttgtccctccaaatccggcctgagctataagagtgtgatcagtttcgtgtgcaggcctgaggccgggccaaccaataggcccatgctcatctccctggacaagcagacatgcactctcttcttctcctggcacacgccgctggcctgcgagcaagcgaccgaatgttccgtgaggaatggaagctctattgttgacttgtctccccttattcatcgcactggtggttatgaggcttatgatgagagtgaggatgatgcctccgataccaaccctgatttctacatcaatatttgtcagccactaaatcccatgcacgnagtgccctgtcctgccggagccgctgtgtgcaaagttcctattgatggtccccccatagatatcggccgggtagcaggaccaccaatactcaatccaatagcaaatgagatttacttgaattttgaaagcagtactccttgcttagcggacaagcatttcaactacacctcgctcatcgcgtttcactgtaagagaggtgtgagcatgggaacgcctaagctgttaaggaccagcgagtgcgactttgtgttcgaatgggagactcctgtcgtctgtcctgatgaagtgaggatggatggctgtaccctgacagatgagcagctcctctacagcttcaacttgtccagcctttccacgagcacctttaaggtgactcgcgactcgcgcacctacagcgttggggtgtgcacctttgcagtcgggccagaacaaggaggctgtaaggacggaggagtctgtctgctctcaggcaccaagggggcatcctttggacggctgcaatcaatgaaactggattacaggcaccaggatgaagcggtcgttttaagttacgtgaatggtgatcgttgccctccagaaaccgatgacggcgtcccctgtgtcttccccttcatattcaatgggaagagctacgaggagtgcatcatagagagcagggcgaagctgtggtgtagcacaactgcggactangacagagaccacgagtggggcttctgcagacactcaaacagctaccggacatccagcatcatatttaagtgtgatgaagatgaggacattgggaggccacaagtcttcagtgaagtgcgtgggtgtgatgtgacatttgagtggaaaacaaaagttgtctgccctccaaagaagttggagtgcaaattcgtccagaaacacaaaacctacgacctgcggctgctctcctctctcaccgggtcctggtccctggtccacancggagtctcgtactatataaatctgtgccagaaaatatataaagggcccctgggctgctctgaaagggccagcatttgcagaaggaccacaactggtgacgtccaggtcctgggactcgttcacacgcagaagctgggtgtcataggtgacaaagttgttgtcacgtactccaaaggttatccgtgtggtggaaataagaccgcatcctccgtgatagaattgacctgtacaaagacggtgggcagacctgcattcaagaggtttgatatcgacagctgcacttactacttcagctgggactcccgggctgcctgcgccgtgaagcctcaggaggtgcagatggtgaatgggaccatcaccaaccctataaatggcaagagcttcagcctcggagatatttattttaagctgttcagagcctctggggacatgaggaccaatggggacaactacctgtatgagatccaactttcctccatcacaagctccagaaacccggcgtgctctggagccaacatatgccaggtgaagcccaacgatcagcacttcagtcggaaagttggaacctctgacaagaccaagtactaccttcaagacggcgatctngatgtcgtgtttgcctcttcctctaagtgcggaaaggataagaccaagtctgtttcttccaccatcttcttccactgtgaccctctggtggaggacgggatccccgagttcagtcacgagactgccgactgccagtacctcttctcttggtacacctcagccgtgtgtcctctgggggtgggctttgacagcgagaatcccggggacgacgggcagatgcacaaggggctgtcagaacggagccaggcagtcggcgcggtgctcagcctgctgctggtggcgctcacctgctgcctgctggccctgttgctctacaagaaggagaggagggaaacagtgataagtaagctgaccacttgctgtaggagaagttccaacgtgtcctacaaatactcaaaggtgaataaggaagaagagacagatgagaatgaaacagagtggctgatggaagagatccagctgcctcctccacggcagggaaaggaagggcaggagaacggccatattaccaccaagtcagtgaaagccctcagctccctgcatggggatgaccaggacagtgaggatgaggttctgaccatcccagaggtgaaagttcactcgggcaggggagctggggcagagagctcccacccagtgagaaacgcacagagcaatgcccttcaggagcgtgaggacgatagggtggggctggtcaggggtgagaaggcgaggaaagggaagtccagctctgcacagcagaagacagtgagctccaccaagctggtgtccttccatgacgacagcgacgaggacctcttacacatctgactccgcagtgcctgcaggggagcacggagccgcgggacagccaagcacctccaaccaaataagacttccactcgatgatgcttctataattttgcctttaacagaaactttcaaaagggaagagtttttgtgatgggggagagggtgaaggaggtcaggccccactccttcctgattgtttacagtcattggaataaggcatggctcagatcggccacagggcggtaccttgtgcccagggttttgccccaagtcctcatttaaaagcataaggccggacgcatctcaaaacagagggctgcattcgaagaaacccttgctgctttagtcccgatagggtatttgaccccgatatattttagcattttaattctctccccctatttattgactttgacaattactcaggtttgagaaaaaggaaaaaaaaacagccaccgtttcttcctgccagcaggggtgtgatgtaccagtttgtccatcttgagatggtgaggctgtcagtgtatggggcagcttccggcgggatgttgaactggtcattaatgtgtcccctgagttggagctcattctgtctcttttctcttttgctttctgtttcttaagggcacacacacgtgcgtgcgagcacacacacacatacgtgcacagggtccccgagtgcctaggttttggagagtttgcctgttctatgcctttagtcaggaatggctgcacctttttgcatgatatcttcaagcctgggcgtacagagcacatttgtcagtatttttgccggctggtgaattcaaacaacctgcccaaagattgatttgtgtgtttgtgtgtgtgtgtgtgtgtgtgtgtgtgtgtgagtggagttgaggtgtcagagaaaatgaattttttccagatttggggtataggtctcatctcttcaggttctcatgataccacctttactgtgcttatttttttaagaaaaaagtgttgatcaaccattcgacctataagaagccttaatttgcacagtgtgtgacttacagaaactgcatgaaaaatcatgggccagagcctcggccntagcattgcacttggcctcatgctggagggaggctgggcgggtacagcgcggaggaggagggaggccaggcgggcatggcgtggaggaggagggaggccgggcggtcacagcatggaggaggagggaggcgctgctggtgttcttattctggcggcagcgcctttcctgccatgtttagtgaatgacttttctcgcattgtagaattgtatatagactctggtgttctattgctgagaagcaaaccgccctgcagcatccctcagcctgtaccggtttggctggcttgtttgatttcaacatgagtgtattttttaaaattgattttnctcttcatttttttttcaatcaactttactgtaatataaagtattcaacaatttcaataaaagataaattattaaaa

SEQ ID NO: 2 - protein, 2491 aa, homo sapiens CI-MPR
MGAAAGRSPHLGPAPARRPQRSLLLLQLLLLVAAPGSTQAQAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKLVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVKEKEDLLCGATDGKKRYDLSALVRHAEPEQNWEAVDGSQTETEKKHFFINICHRVLQEGKARGCPEDAAVCAVDKNGSKNLGKFISSPMKEKGNIQLSYSDGDDCGHGKKIKTNITLVCKPGDLESAPVLRTSGEGGCFYEFEWRTAAACVLSKTEGENCTVFDSQAGFSFDLSPLTKKNGAYKVETKKYDFYINVCGPVSVSPCQPDSGACQVAKSDEKTWNLGLSNAKLSYYDGMIQLNYRGGTPYNNERHTPRATLITFLCDRDAGVGFPEYQEEDNSTYNFRWYTSYACPEEPLECVVTDPSTLEQYDLSSLAKSEGGLGGNWYAMDNSGEHVTWRKYYINVCRPLNPVPGCNRYASACQMKYEKDQGSFTEVVSISNLGMAKTGPVVEDSGSLLLEYVNGSACTTSDGRQTTYTTRIHLVCSRGRLNSHPIFSLNWECVVSFLWNTEAACPIQTTTDTDQACSIRDPNSGFVFNLNPLNSSQGYNVSGIGKIFMFNVCGTMPVCGTILGKPASGCEAETQTEELKNWKPARPVGIEKSLQLSTEGFITLTYKGPLSAKGTADAFIVRFVCNDDVYSGPLKFLHQDIDSGQGIRNTYFEFETALACVPSPVDCQVTDLAGNEYDLTGLSTVRKPWTAVDTSVDGRKRTFYLSVCNPLPYIPGCQGSAVGSCLVSEGNSWNLGVVQMSPQAAANGSLSIMYVNGDKCGNQRFSTRITFECAQISGSPAFQLQDGCEYVFIWRTVEACPVVRVEGDNCEVKDPRHGNLYDLKPLGLNDTIVSAGEYTYYFRVCGKLSSDVCPTSDKSKVVSSCQEKREPQGFHKVAGLLTQKLTYENGLLKMNFTGGDTCHKVYQRSTAIFFYCDRGTQRPVFLKETSDCSYLFEWRTQYACPPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHAVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCPPKKLECKFVQKHKTYDLRLLSSLTGSWSLVHNGVSYYINLCQKIYKGPLGCSERASICRRTTTGDVQVLGLVHTQKLGVIGDKVVVTYSKGYPCGGNKTASSVIELTCTKTVGRPAFKRFDIDSCTYYFSWDSRAACAVKPQEVQMVNGTITNPINGKSFSLGDIYFKLFRASGDMRTNGDNYLYEIQLSSITSSRNPACSGANICQVKPNDQHFSRKVGTSDKTKYYLQDGDLDVVFASSSKCGKDKTKSVSSTIFFHCDPLVEDGIPEFSHETADCQYLFSWYTSAVCPLGVGFDSENPGDDGQMHKGLSERSQAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 3 - DNA, 2454 bp, homo sapiens CD-MPR
ttccggttcccagagtggggcacagcgaggcgctagggggaacgctggcctctgaaactagctctgggaccggggtctgcggccggcccctagctggccccgtctcccatccccagaagggtattcactggggattctgagctttggctactccagtttcccacgacacgatgttccctttctacagctgctggaggactggactgctactactactcctggctgtggcagtgagagaatcctggcagacagaagaaaaaacttgcgacttggtaggagaaaagggtaaagagtcagagaaagagttggctctagtgaagaggctgaaaccactgtttaataaaagctttgagagcactgtgggccagggttcagacacatacatctacatcttcagggtgtgccgggaagctggcaaccacacttctggggcaggcctggtgcaaatcaacaaaagtaatgggaaggagacagtggtagggagactcaacgagactcacatcttcaacggaagtaattggatcatgctgatctataaagggggtgatgaatatgacaaccactgtggcaaggagcagcgtcgtgcagtggtgatgatctcctgcaatcgacacaccctagcggacaattttaaccctgtgtctgaggagcgtggcaaagtccaagattgtttctacctctttgagatggatagcagcctggcctgttcaccagagatctcccacctcagtgtgggttccatcttacttgtcacgtttgcatcactggttgctgtttatgttgttggggggttcctataccagcgactggtagtgggagccaaaggaatggagcagtttccccacttagccttctggcaggatcttggcaacctggtagcagatggctgtgactttgtctgccgttctaaacctcgaaatgtgcctgcagcatatcgtggtgtgggggatgaccagctgggggaggagtcagaagaaagggatgaccatttattaccaatgtagattgcactttatatgtccagcctcttcctcagtcccccaaaccaaagctacacagccagatttctcaagcagtctcaactccagtccctcatctcacccttactattgctcttgctttccagtttgcttttgatttgcatcttctcactagtaaaactgccttccctttgttccttattttctgttttttctctagagaggtacagttgtaagtcagagttaatataatagggcctgtgaaaacagaggcttttgcattgtctcttgacatcagaagttacaataggcatatgggcaaaatggtgtagcaggctcactggccgtttgttttttaaacacattttcacaagtttttgagacactggatttctttaattaaaaaaaaaatgccaagaaacattatttatacagggttgattgctttcatgttgttattctgtaccctatagtagcctccatgagaatctggtatttcttgctgcttggaactactttgcagtgattacttggttgcagtccaagtactctcgtttagtctgagcctggagatgttctagacttgcttctcccacctctgagattaggacaggaaaaatgtgaaatttcccaattacaggattatacggtaccatcacatcatttgtggaaattggggtgactgtatagctgggattgggctaaggactgtggtcttatctgtccacatacagccaaaatgcctatccagaaatccagttcgttggaaaggaaaattggtactcctgtgccacaggggttccagaaaagggaagtcactttaccttgcggtggtgggatcctgatgtctttcatccatttgtagtaaaagctggtaaagcttttcttactcctggttccctaccagtatttctaaacatgtcgcactttctccacaggcatgtggttttgacctttttttcaatcttctagaaagggaacggaagcagaagtgggacatcgagggctctgctgtcctctgcgctgggtgtggaatgctgctgcacctgtcccttctgctggctcagggaagtgtcttcttgcccacatttctgtggggaaaggtttttaatcctctgatgcttccatcttcctgtttaggccatgtgcccagaaacctggactgatctttctttaatagtgaacccctgggccactgaagagtaacatggctccactggacacaaaagagggatggaatcaacaggcagggggccttttataagccttaggaaaagaaaatgaaactatttcatctttggacttttcaatactattggagtgatttttttctttctaaacagggaaaataatgttacaaaagcatcttttttgttatttgtttgcatccctcccccacaccctggtgttttaaaatgaagaaaaaaaaccatcaccttttgtacaaaaactcttaatgattaaaaaacaaaca

SEQ ID NO: 4 - protein, 277 aa, homo sapiens CD-MPR
MFPFYSCWRTGLLLLLLAVAVRESWQTEEKTCDLVGEKGKESEKELALVKRLKPLFNKSFESTVGQGSDTYIYIFRVCREAGNHTSGAGLVQINKSNGKETVVGRLNETHIFNGSNWIMLIYKGGDEYDNHCGKEQRRAVVMISCNRHTLADNFNPVSEERGKVQDCFYLFEMDSSLACSPEISHLSVGSILLVTFASLVAVYVVGGFLYQRLVVGAKGMEQFPHLAFWQDLGNLVADGCDFVCRSKPRNVPAAYRGVGDDQLGEESEERDDHLLPM

SEQ ID NO: 5 - DNA, 879 bp, homo sapiens grB
agcagctccaaccagggcagccttcctgagaagatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctct

SEQ ID NO: 6 - protein, 247 aa, homo sapiens grB
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY